(19) United States
(12) Patent Application Publication
(10) Pub. No.: US 2025/0084392 A1
(43) Pub. Date: Mar. 13, 2025

(54) STRATEGIES FOR DIRECT RECRUITMENT OF REPAIR TEMPLATES TO CRISPR NUCLEASES

(71) Applicant: INSCRIPTA, INC., Boulder, CO (US)

(72) Inventors: Brian Chaikind, Pleasanton, CA (US); Christopher Lim, Pleasanton, CA (US)

(21) Appl. No.: 18/725,912

(22) PCT Filed: Jan. 4, 2023
(86) PCT No.: PCT/US2023/060106

§ 371 Date: Jul. 1, 2024

Related U.S. Application Data
(60) Provisional application No. 63/296,461, filed on Jan. 4, 2022.

(51) Int. Cl. C12N 9/22; C12N 15/11; C12N 15/90

(52) U.S. Cl. CPC C12N 9/22; C12N 15/111; C12N 15/907; C07K 2319/20; C12N 2310/20

(57) ABSTRACT

This invention relates to compositions of matter, methods and instruments for directly recruiting repair templates to CRISPR nucleases to stimulate homology-directed repair. Molecular “tethers” are described which result in an increase in the local concentration of repair templates at the site of the double-strand break made by a nuclease, thereby enhancing the rate of homology directed repair and suppressing undesired edits.

### INCORPORATION OF SEQUENCE LISTING

This application claims the benefit of U.S. Provisional Application No. 63/296,461, filed Jan. 4, 2022, which is incorporated by reference in its entirety herein. A sequence listing contained in the file named “P35287WO00_SL.xml” which is 3,462 bytes (measured in MS-Windows®) and created on Jan. 4, 2023, is filed electronically herewith and incorporated by reference in its entirety.

### FIELD OF THE INVENTION

This invention relates to compositions of matter, methods and instruments for directly recruiting repair templates to CRISPR nucleases to stimulate homology-directed repair while simultaneously increasing protospacer adjacent motif (PAM) availability.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the methods referenced herein do not constitute prior art under the applicable statutory provisions.

CRISPR nucleases are programmable nucleases that generate genomic lesions that can be repaired through a variety of cellular mechanisms. The predominant repair pathway is termed non-homologous end joining (NHEJ) in which small insertions/deletions (indels) are incorporated near the break site, disrupting genomic coding regions by frameshift mutations. In contrast to NHEJ, homology directed repair (HDR) utilizes a donor DNA (e.g., a repair template) as a template for repair, inserting “correct” genetic information to repair the genetic lesion. With the advent of CRISPR gene editing for both research and therapeutic purposes, various methods have been used to suppress NHEJ and/or favor-IDR to enable precision editing al a given genomic locus.

There is thus a need in the art of CRISPR nuclease editing for improved methods, compositions, modules and instruments for suppressing NHEJ and favoring HDR. The present disclosure addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In one aspect, this disclosure provides, and includes, a system comprising: (i) a fusion polypeptide comprising first and second orthogonal nucleases and further comprising or coupled to a recruiting moiety, (ii) a repair template comprising or coupled with a binding moiety, and optionally (iii) one or more guide RNAs; wherein the recruiting moiety recognizes the binding moiety and forms a binding pair.

In one aspect, this disclosure provides, and includes, a system comprising: (i) an RNA-guided nuclease, (ii) a guide RNA comprising or coupled to a recruiting moiety, and (iii) a repair template molecule comprising or coupled with a binding moiety; wherein the recruiting moiety recognizes the binding moiety and forms a binding pair. These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1A is an overview of DNA repair pathways following a CRISPR nuclease-induced double-stranded break.

FIG. 1B is a simplified block diagram of an example of a method for editing live cells via editing cassettes comprising a binding moiety of a binding pair and fusion enzyme constructs comprising two orthogonal nucleases and a recruiting moiety of the binding pair.

FIG. 1C depicts recruitment of an editing cassette with a binding moiety (“ECBM”), here biotin, recruited to a fusion enzyme construct comprising orthogonal nucleases and a recruitment moiety, here streptavidin.

FIG. 1D depicts recruitment of an editing cassette with a binding moiety (“ECBM”), here a HUH recognition sequence, recruited to a fusion enzyme construct comprising orthogonal nucleases and a recruitment moiety, here a HUH-tag.

FIG. 1E depicts recruitment of an editing cassette with a binding moiety (“ECBM”), here uracilated DNA, recruited to a fusion enzyme construct comprising orthogonal nucleases and a recruitment moiety, here Udg variants.

FIG. 1F depicts recruitment of an editing cassette with a binding moiety (“ECBM”), here the synthesized repair template made by a gRNA-retron fusion, recruited to a fusion enzyme construct comprising orthogonal nucleases and a recruitment moiety, here the retron.

FIG. 1G depicts using an RNA/DNA hetero-G-quadruplex (GQ) structure non-covalently linking a single guide RNA (sgRNA) and a single-stranded oligonucleotide donor DNA (ssODN) for recruitment of donor DNA to the site of lesion to enhance precise genome editing via HDR.

FIG. 1H depicts using an RNA/DNA heteroduplex structure comprising a heteroduplex barcode sequence to non-covalently link the ssODN and sgRNA for recruitment of donor DNA to the site of lesion to enhance precise genome editing via HDR.

FIGS. 2A-2C depict three different views of an example of an automated multi-module cell processing instrument for performing fusion enzyme construct editing.

FIG. 2D is a simplified process diagram of an aspect of an example of an automated multi-module cell processing instrument comprising a solid wall selection/singulation/growth/induction/editing/normalization device (such as shown in FIGS. 2A-2C) for recursive cell editing in a system using a fusion enzyme construct and an editing cassette comprising a binding moiety.

FIGS. 3A-3C depict various views and components of an example of a bioreactor module included in an integrated instrument useful for growing and transfecting cells, particularly mammalian cells, for performing fusion enzyme construct editing.

FIGS. 3D and 3E depict an example of an integrated instrument comprising the bioreactor of FIGS. 3A-3C for growing and transfecting cells for performing fusion enzyme construct editing.

FIG. 4A depicts an example of a workflow employing microcarrier-partitioned delivery for cells for performing fusion enzyme construct editing of mammalian cells grown in suspension.

FIG. 4B depicts an option for growing, passaging, transfecting and editing iPSCs (induced pluripotent stem cells) involving sequential transduction and transfection of editing cassettes and fusion enzyme constructs.

FIG. 4C depicts an example of a workflow employing microcarrier-partitioned delivery for performing fusion enzyme construct editing of mammalian cells.

FIG. 4D depicts an alternative workflow employing microcarrier-partitioned delivery for performing fusion enzyme construct editing of mammalian cells.

It should be understood that the drawings are not necessarily to scale, and that like reference numbers refer to like features.

### DETAILED DESCRIPTION

All the functionalities described in connection with one aspect are intended to be applicable to the additional aspects described herein except where expressly stated or where the feature or function is incompatible with the additional aspects. For example, where a given feature or function is expressly described in connection with one aspect but not expressly mentioned in connection with an alternative aspect, it should be understood that the feature or function may be deployed, utilized, or implemented in connection with the alternative aspect unless the feature or function is incompatible with the alternative aspect.

The practice of the techniques described herein may employ, unless otherwise indicated, conventional techniques and descriptions of organic chemistry, polymer technology, molecular biology (including recombinant techniques), cell biology, biochemistry and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent procedures can, of course, also be used. Such techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W. H. Freeman, New York N.Y.; Gait, “Oligonucleotide Synthesis: A Practical Approach” (1984), IRL Press, London; Nelson and Cox (2000), Lehninger, Principles of Biochemistry 3rd Ed., W. H. Freeman Pub., New York, N.Y.; Berg, et al. (2002) Biochemistry, 5th Ed., W. H. Freeman Pub., New York, N.Y.; all of which are herein incorporated in their entirety by reference for all purposes. CRISPR-specific techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes. Note that as used herein and in the appended claims, the singular forms “a,” “an,” and “the” include plural references unless the context clearly dictates otherwise. Thus, for example, reference to “an oligonucleotide” refers to one or more oligonucleotides, and reference to “an automated system” includes reference to equivalent steps and methods for use with the system known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit aspects of the present disclosure to any particular orientation or configuration. Furthermore, terms such as “first,” “second,” “third,” etc., merely identify, one of a number of portions, components, steps, operations, functions, and/or points of reference as disclosed herein, and likewise do not necessarily limit aspects of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference herein in their entireties.

When a range of numbers is provided herein the range is understood to be inclusive of the edges of the range as well as any number between the defined edges of the range.

For example, “between 1 and 10” includes any number between 1 and 10, as well as the number 1 and number 10.

The term “about” means plus or minus 10% of the numerical value of the number with which it is being used. For example, “about 100” refers to numbers between (and including) 90 and 110.

When a grouping of alternatives is presented, any and all combinations of the members that make up that grouping of alternatives is specifically envisioned. For example, if an item is selected from a group consisting of A, B, C, and D, the inventors specifically envisions each alternative individually (e.g. A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

In the following description, numerous specific details are set forth to provide a more thorough understanding of the present invention. However, it will be apparent to one of ordinary skill in the art that the present invention may be practiced without one or more of these specific details. In other instances, well-known features and procedures well known to those skilled in the art have not been described in order to avoid obscuring the invention.

The term “binding moiety” refers to a moiety that binds a “recruiting moiety.” A “binding pair” comprises a binding moiety and a recruiting moiety. In some aspects, a moiety of a binding pair is located on or coupled to a repair template. In some aspects, a moiety of a binding pair is located on or coupled to a fusion enzyme construct comprising two orthogonal nucleases. In some aspects, a moiety of a binding pair is located on a guide RNA (gRNA). In some aspects, a recruiting moiety is a nucleic acid sequence that is located on a fusion enzyme construct comprising two orthogonal nucleases. In some aspects, a recruiting moiety is a nucleic acid, a polypeptide, a chemical modification, or any combination thereof. In some aspects, a recruiting moiety is further chemically and/or covalently modified. In some aspects, a binding moiety is a nucleic acid, a polypeptide, a chemical modification or any combination thereof. In some aspects, a binding moiety is further chemically and/or covalently modified. In some aspects, the formation of the binding pair is via non-covalent interactions. In some aspects, the formation of the binding pair is via covalent interactions.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. The terms “percent complementarity” or “percent complementary” as used herein in reference to two nucleotide sequences is similar to the concept of percent identity but refers to the percentage of nucleotides of a query sequence that optimally base-pair or hybridize to nucleotides in a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (e.g., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%. In general, a nucleic acid includes a nucleotide sequence described as having a “percent complementarity” or being a “percent complementary” to a specified second nucleotide sequence. For example, a nucleotide sequence may have 70%, 80%, 90%, 95%, 99%, or 100% complementarity to a specified second nucleotide sequence, indicating that, for example, 7 of 10, 8 of 10, 9 of 10, 19 of 20, 99 of 100, or 10 of 10 nucleotides, respectively, of a sequence are complementary to the specified second nucleotide sequence. For example, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

The terms “editing cassette” or “CREATE cassette” refer to a nucleic acid molecule comprising a coding sequence for transcription of a gRNA covalently linked to a coding sequence for transcription or reverse transcription of a repair template. For additional information regarding editing cassettes, see U.S. Pat. Nos. 9,982,278; 10,266,849; 10,240,167; 10,351,877; 10,364,442; 10,435,715; 10,465,207; 10,669,559; 10,771,284; 10,731,498; and 11,078,498. In the present compositions and methods, the editing cassette further comprises a binding moiety of a binding pair (an “editing cassette with binding moiety” or “ECBM”). In the present compositions and methods, the editing cassette encodes a recruitment construct. As used herein a “recruitment construct” refers to a construct comprising, in any order, a moiety of a binding pair and a repair template (e.g., a transcription or reverse transcription of the repair template). In some aspects, the recruitment construct comprises, in any order, a gRNA, a binding moiety of a binding pair, and a repair template (e.g., a transcription or reverse transcription of the repair template). In some aspects, the recruitment construct further comprises a barcode. In some aspects, the gRNA and repair template (e.g., a transcription or reverse transcription of the repair template) form an RNA/DNA hetero-G quadruplex. In some aspects, the gRNA and repair template (e.g., a transcription or reverse transcription of the repair template) form an RNA/DNA heteroduplex further comprising a heteroduplex barcode. In some aspects of the present disclosure, “editing cassette” and “recruitment construct” may be used interchangeably, for instance, when referring to the “editing cassette with binding moiety” or “ECBM” which refers to a recruitment construct comprising a binding moiety of a binding pair.

As used herein, the phrases “fusion enzyme construct” or “dual orthogonal enzyme construct” or “CRISPR fusion enzyme construct” refer to a CRISPR nuclease that has been engineered to comprise coding regions for two orthogonal nucleases and a recruitment moiety of a binding pair, or the translated proteins and recruitment moiety construct, or constructs that can assemble together in a multi-protein complex, wherein the recruitment moiety binds the binding moiety portion of the ECBM. The terms “guide nucleic acid” or “guide RNA” or “gRNA” or “single guide RNA” or “sgRNA” refer to a polynucleotide comprising (1) a guide sequence capable of hybridizing to a genomic target locus, and (2) a scaffold sequence capable of interacting or complexing with a nucleic acid-guided nuclease.

“Homology” or “identity” or “similarity” refers to sequence similarity between two peptides or, more often in the context of the present disclosure, between two nucleic acid molecules. The term “homologous region” refers to a region on the gRNA or repair template with a certain degree of homology with the target DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

The terms “percent identity” or “percent identical” as used herein in reference to two or more nucleotide or amino acid sequences is calculated by (i) comparing two optimally aligned sequences (nucleotide or amino acid) over a window of comparison (the “alignable” region or regions), (ii) determining the number of positions at which the identical nucleic acid base (for nucleotide sequences) or amino acid residue (for proteins and polypeptides) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to amino acids it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

For optimal alignment of sequences to calculate their percent identity, various pair-wise or multiple sequence alignment algorithms and programs are known in the art, such as ClustalW or Basic Local Alignment Search Tool® (BLAST™), etc., that can be used to compare the sequence identity or similarity between two or more nucleotide or amino acid sequences. Although other alignment and comparison methods are known in the art, the alignment and percent identity between two sequences (including the percent identity ranges described above) can be as determined by the ClustalW algorithm, see, e.g., Chenna et al., “Multiple sequence alignment with the Clustal series of programs,” Nucleic Acids Research 31: 3497-3500 (2003); Thompson et al., “Clustal W: Improving the sensitivity of progressive multiple sequence alignment through sequence weighting, position-specific gap penalties and weight matrix choice,” Nucleic Acids Research 22: 4673-4680 (1994); Larkin M A et al., “Clustal W and Clustal X version 2.0,” Bioinformatics 23: 2947-48 (2007); and Altschul et al. “Basic local alignment search tool.” J. Mol. Biol. 215:403-410 (1990), the entire contents and disclosures of which are incorporated herein by reference.

The terms “fusion enzyme editing components” or “CRISPR editing components” refer to one or both of a fusion enzyme construct and an editing cassette comprising a binding moiety (“ECBM”).

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (e.g. chromosome) and may still have interactions resulting in altered regulation.

The term “orthogonal nucleases” refers to CRISPR nucleases where the gRNAs that complex with the CRISPR nucleases are orthogonal. The structural regions of the gRNAs are only recognized by one of the two or more nucleases (e.g., Cas proteins). Thus, by altering the sequence and structure of the gRNAs, each nuclease (e.g., Cas protein) only binds and is guided by its respective gRNA partner.

A “PAM mutation” refers to one or more edits to a target sequence that removes, mutates, or otherwise renders inactive a PAM (i.e., protospacer adjacent motif) or spacer region in the target sequence.

A “regulatory sequence” or “regulatory region” refers to the region of a gene where RNA polymerase and other accessory transcription modulator proteins (e.g., transcription factors) bind and interact to control transcription of the gene. Non-limiting examples of regulatory sequences or regions include promoters, enhancers, and terminators. Regulatory sequences or regions are capable of increasing or decreasing gene expression. As a result, these elements can control net protein expression from the gene.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA. Promoters may be constitutive or inducible. A “pol II promoter” is a regulatory sequence that is bound by RNA polymerase II to catalyze the transcription of DNA. In some aspects, a promoter is an endogenous promoter, synthetically produced, varied, or derived from a known or naturally occurring promoter sequence or other promoter sequence. In some aspects, a promoter is a constitutive promoter. In some aspects, a promoter is an inducible promoter. In some aspects, a promoter is a heterologous promoter.

A “terminator” or “terminator sequence” refers to a DNA regulatory region of a gene that signals termination of transcription of the gene to an RNA polymerase. Without being limiting, terminators cause transcription of an operably linked nucleic acid molecule to stop.

A “coding sequence” or “coding region” refers to the region of a gene's DNA or RNA which codes for a gene product (e.g., a protein). In DNA, the coding region of a gene is flanked by the promoter sequence on the 5′ end of the template strand and the termination sequence on the 3′ end. After transcription, the coding region in an mRNA is flanked by the 5′ untranslated region (5′-UTR) and 3′ untranslated region (3′-UTR), the 5′ cap, and poly-A tail.

A “non-coding sequence” or “non-coding region” refers to the region of a gene's DNA which does not code for a protein. However, some non-coding DNA is transcribed into functional non-coding RNA molecules (e.g., transfer RNA, microRNA, siRNA, piRNA, ribosomal RNA, and regulatory RNAs). Other functional non-coding DNA include, for example, regulatory sequences of a gene that control its expression.

As used herein “gene product” refers to a biochemical material, either RNA or protein, resulting from expression of a gene. In some aspects, a gene product is an RNA molecule, e.g., transfer RNA, microRNA, siRNA, piRNA, ribosomal RNA, or regulatory RNA. In some aspects, the gene product is a protein. In some aspects, the gene product is an enzyme. In some aspects, the gene product is a membrane protein. In some aspects, the gene product is a protein involved in the expression of a gene. In some aspects, the gene product is a transcription factor. In some aspects, the gene product is a coactivator protein. In some aspects, the gene product is a corepressor protein. In some aspects, the gene product is a chromatin-binding protein. As used herein, the terms “protein,” “peptide,” and “polypeptide” are used interchangeably herein and refer to a polymer of amino acid residues. In some aspects, proteins are made up entirely of amino acids transcribed by any class of any RNA polymerase I, II or III.

As used herein the term “repair template” or “donor” refers to a nucleic acid that is designed to serve as a template (including a desired edit) to be incorporated into target DNA via HDR. In the present aspects, the repair template comprises sufficient flanking homology around the site of a double-strand break in the genomic target locus and a region near the break site that encodes the precise edit. Alternatively, the repair template may also have no homology around the site of one or more double stranded break, enabling NHEJ or ligase-dependent insertion or replacement at the site of DSB(s).

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 may be employed. In other aspects, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+ cells); CD24 cell surface antigen in hematopoietic stem cells; human CAD gene to confer resistance to N-phosphonoacetyl-L-aspartate (PALA); human multi-drug resistance-1 (MDR-1; P-glycoprotein surface protein selectable by increased drug resistance or enriched by FACS); human CD25 (IL-2α; detectable by Mab-FITC); Methylguanine-DNA methyltransferase (MGMT; selectable by carmustine); rhamnose; and Cytidine deaminase (CD; selectable by Ara-C). In some aspects, a selectable marker comprises an antibiotic resistance gene. In some aspects, a selectable marker comprises a puromycin resistance gene. “Selective medium” as used herein refers to cell growth medium to which has been added a chemical compound or biological moiety that selects for or against selectable markers. A “locus” refers to a fixed position in a genome. In some aspects, a locus comprises a coding region. In some aspects, a locus comprises a non-coding region. In some aspects, a locus comprises a gene. In an aspect, a locus comprises at least 1 nucleotide. In an aspect, a locus comprises at least 10 nucleotides. In an aspect, a locus comprises at least 25 nucleotides. In an aspect, a locus comprises at least 50 nucleotides. In an aspect, a locus comprises at least 100 nucleotides. In an aspect, a locus comprises at least 250 nucleotides. In an aspect, a locus comprises at least 500 nucleotides. In an aspect, a locus comprises at least 1000 nucleotides. In an aspect, a locus comprises at least 2500 nucleotides. In an aspect, a locus comprises at least 5000 nucleotides.

The terms “target DNA sequence”, “target region”, “cellular target sequence”, or “genomic target locus” refer to any locus in vitro or in vivo, or in a nucleic acid (e.g., genome or episome) of a cell or population of cells, in which a change of at least one nucleotide is desired using a nucleic acid-guided nuclease editing system. The cellular target sequence can be a genomic locus or extrachromosomal locus. The target genomic DNA sequence comprises the edit region or edit locus. In some aspects, a target locus comprises a gene, including its regulatory regions and coding regions. In some aspects, a target locus comprises a regulatory region of a gene, e.g., a promoter region or a terminator region.

The term “gene” refers to a nucleic acid region which includes a coding region operably linked to a suitable regulatory region capable of regulating the expression of a gene product (e.g., a polypeptide or functional RNA) in some manner. Genes include untranslated regulatory regions (e.g., promoters, enhancers, repressors, etc.) in the DNA before (upstream) and after (downstream) the coding region (open reading frame, ORF), and, where applicable, intervening sequences (e.g., introns) between individual coding regions (e.g., exons).

The term “variant” may refer to a polypeptide or polynucleotide that differs from a reference polypeptide or polynucleotide. A typical variant of a polypeptide differs in amino acid sequence from another reference polypeptide. Generally, differences may be limited so that the sequences of the reference polypeptide and the variant are closely similar overall (e.g., at least 90% identical) and, in many regions, identical. A variant and reference polypeptide may differ in amino acid sequence by one or more modifications (e.g., substitutions, additions, and/or deletions). A variant of a polypeptide may be a conservatively modified variant (e.g., at least 95% identical to the reference polypeptide). A substituted or inserted amino acid residue may or may not be one encoded by the genetic code (e.g., anon-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, BACs, YACs, PACs, synthetic chromosomes, and the like. As used herein, the phrase “engine vector” comprises a coding sequence for a fusion enzyme construct to be used in the CREATE fusion editing systems and methods of the present disclosure. As used herein the phrase “editing vector” comprises a repair template-including an alteration to the cellular target sequence that prevents nuclease binding at a PAM or spacer in the cellular target sequence after editing has taken place-covalently linked to a coding sequence for a gRNA. The editing vector may also and preferably does comprise a selectable marker and/or a barcode, and/or, as described herein, an RNA stabilization moiety. In some aspects, the engine vector and editing vector may be combined; that is, all fusion enzyme construct editing components may be found on a single vector. Further, the engine and editing vectors comprise control sequences operably linked to, e.g., the fusion enzyme construct coding sequence and the editing cassette.

As used herein, a “mutation” refers to an inheritable genetic modification introduced into a gene to alter the expression or activity of a product encoded by the gene. In some aspects, “mutation,” “modification,” and “edit” may be used interchangeably in the present disclosure. In some aspects, a modification can be in any sequence region of a gene, for example, in a promoter, 5′ UTR, exon, 3′ UTR, or terminator region. In some aspects, a modification can be in the regulatory region of a gene. In some aspects, a modification can be in the coding region of a gene. In some aspects, a modification reduces, inhibits, or eliminates the expression or activity of a gene product. In some aspects, a modification increases, elevates, strengthens, or augments the expression or activity of a gene product.

In some aspects, a mutation, or modification is a “non-natural” or “non-naturally occurring” mutation or modification. As used herein, a “non-natural” or “non-naturally occurring” mutation or modification refers to a non-spontaneous mutation or modification generated via human intervention, and does not correspond to a spontaneous mutation or modification generated without human intervention. Non-limiting examples of human intervention include mutagenesis (e.g., chemical mutagenesis, ionizing radiation mutagenesis) and targeted genetic modifications (e.g., nucleic-acid guided nuclease-based methods, CREATE fusion-based methods, CRISPR-based methods, TALEN-based methods, zinc finger-based methods). Non-natural mutations or modifications and non-naturally occurring mutations or modifications do not include spontaneous mutations that arise naturally (e.g., via aberrant DNA replication).

Several types of mutations or modifications are known in the art. In some aspects, a mutation or modification comprises an insertion. An “insertion” refers to the addition of one or more nucleotides or amino acids to a given polynucleotide or amino acid sequence, respectively, as compared to an endogenous reference polynucleotide or amino acid sequence.

In some aspects, a mutation or modification comprises a deletion. A “deletion” refers to the removal of one or more nucleotides or amino acids to a given polynucleotide or amino acid sequence, respectively, as compared to an endogenous reference polynucleotide or amino acid sequence.

In some aspects, a mutation or modification comprises a substitution or a swap. A “substitution” or “swap” refers to the replacement of one or more nucleotides or amino acids to a given polynucleotide or amino acid sequence, respectively, as compared to an endogenous reference polynucleotide or amino acid sequence. In some aspects, a “substitution allele” refers to a nucleic acid sequence at a particular locus comprising a substitution.

In some aspects, a mutation or modification comprises an inversion. An “inversion” refers to when a segment of a polynucleotide or amino acid sequence is reversed end-to-end. In some aspects, a mutation or modification provided herein comprises a mutation selected from the group consisting of an insertion, a deletion, a substitution, and an inversion. In some aspects, a mutation or modification provided herein comprises an insertion. In some aspects, a mutation or modification provided herein comprises a deletion. In some aspects, a mutation or modification provided herein comprises a substitution. In some aspects, a mutation or modification provided herein comprises an inversion.

In some aspects, a mutation or modification comprises one or more mutation types selected from the group consisting of a nonsense mutation, a missense mutation, a frameshift mutation, a splice-site mutation, and any combinations thereof. As used herein, a “nonsense mutation” refers to a mutation to a nucleic acid sequence that introduces a premature stop codon to an amino acid sequence by the nucleic acid sequence. As used herein, a “missense mutation” refers to a mutation to a nucleic acid sequence that causes a substitution within the amino acid sequence encoded by the nucleic acid sequence. As used herein, a “frameshift mutation” refers to an insertion or deletion to a nucleic acid sequence that shifts the frame for translating the nucleic acid sequence to an amino acid sequence. A “splice-site mutation” refers to a mutation in a nucleic acid sequence that causes an intron to be retained for protein translation, or, alternatively, for an exon to be excluded from protein translation. Splice-site mutations can cause nonsense, missense, or frameshift mutations. Mutations or modifications in coding regions of genes (e.g., exonic mutations) can result in a truncated protein or polypeptide when a mutated messenger RNA (mRNA) is translated into a protein or polypeptide. In some aspects, this disclosure provides a mutation that results in the truncation of a protein or polypeptide. As used herein, a “truncated” protein or polypeptide comprises at least one fewer amino acid as compared to an endogenous control protein or polypeptide. For example, if endogenous Protein A comprises 100 amino acids, a truncated version of Protein A can comprise between 1 and 99 amino acids.

Without being limited by any scientific theory, one way to cause a protein or polypeptide truncation is by the introduction of a premature stop codon in an mRNA transcript of an endogenous gene. In some aspects, this disclosure provides a mutation that results in a premature stop codon in an mRNA transcript of an endogenous gene. As used herein, a “stop codon” refers to a nucleotide triplet within an mRNA transcript that signals a termination of protein translation. A “premature stop codon” refers to a stop codon positioned earlier (e.g., on the 5′-side) than the normal stop codon position in an endogenous mRNA transcript. Without being limiting, several stop codons are known in the art, including “UAG,” “UAA,” “UGA,” “TAG,” “TAA,” and “TGA.” In some aspects, multiple (e.g., 2, 3, 4, 5, 6, 7, 8, 9, 10, or more than 10) premature stop codons are introduced.

In some aspects, a mutation or modification provided herein comprises a null mutation. As used herein, a “null mutation” refers to a mutation that confers a decreased function or complete loss-of-function for a protein encoded by a gene comprising the mutation, or, alternatively, a mutation that confers a decreased function or complete loss-of-function for a small RNA encoded by a genomic locus. A null mutation can cause lack or decrease of mRNA transcript production, small RNA transcript production, protein function, or a combination thereof. As used herein, a “null allele” refers to a nucleic acid sequence at a particular locus where a null mutation has conferred a decreased function or complete loss-of-function to the allele.

In some aspects, a “synonymous edit” or “synonymous substitution” is the substitution of one base for another in an exon of a gene coding for a protein, such that the produced amino acid sequence is not modified. This is possible because the genetic code is “degenerate”, meaning that some amino acids are coded for by more than one three-base-pair codon; since some of the codons for a given amino acid differ by just one base pair from others coding for the same amino acid, a mutation that replaces the “normal” base by one of the alternatives will result in incorporation of the same amino acid into the growing polypeptide chain when the gene is translated.

In some aspects, “codon optimization” refers to experimental approaches designed to improve the codon composition of a recombinant gene based on various criteria without altering the amino acid sequence. This is possible because most amino acids are encoded by more than one codon. Codon optimization may be used to improve gene expression and increase the translation efficiency of a gene of interest by accommodating for codon bias of the host organism. In some aspects, a nucleic acid molecule provided herein encodes a polypeptide that is codon optimized for a prokaryote. In some aspects, a nucleic acid molecule provided herein encodes a polypeptide that is codon optimized for a eukaryote. In some aspects, a nucleic acid molecule provided herein encodes a polypeptide that is codon optimized for a mammalian cell. In some aspects, a nucleic acid molecule provided herein encodes a polypeptide that is codon optimized for an archaeal cell.

In some aspects, a mutation or modification provided herein can be positioned in any part of a gene. In some aspects, a mutation or modification provided herein can be positioned in the coding region of a gene. In some aspects, a mutation or modification provided herein can be positioned in the non-coding region of a gene. In some aspects, a mutation or modification provided herein can be positioned in the regulatory region of a gene. In some aspects, a mutation or modification provided herein is positioned within an exon of a gene. In some aspects, a mutation or modification provided herein is positioned within an intron of a gene. In a further aspect, a mutation or modification provided herein is positioned within a 5′-untranslated region (UTR) of a gene. In still another aspect, a mutation or modification provided herein is positioned within a 3′-UTR of a gene. In yet another aspect, a mutation or modification provided herein is positioned within a promoter of a gene. In yet another aspect, a mutation or modification provided herein is positioned within a terminator of a gene.

In some aspects, the nuclease includes a MAD-series nuclease, nickase, or a variant (e.g., orthologue) thereof. In some aspects, the nuclease includes a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, MAD20, MAD2001, MAD2007, MAD2008, MAD2009, MAD2011, MAD2017, MAD2019, MAD297, MAD298, MAD299, or other MAD-series nuclease, nickase, variants thereof, and/or combinations thereof.

In some aspects, the nuclease is an RNA-guided nuclease. In some aspects, the nuclease is a CRISPR nuclease. In some aspects, the nuclease is a wildtype nuclease. In some aspects, the nuclease is a variant that recognizes an alternative PAM sequence compared to the canonical PAM sequence. In some aspects, the nuclease is a variant that retains wildtype enzymatic activity. In some aspects, the nuclease is a variant that has increased enzymatic activity. In some aspects, the nuclease is a variant that has reduced enzymatic activity. In some aspects, the nuclease is a variant that has no enzymatic activity (e.g. dead enzyme with respect to cleavage activity). In some aspects, the nuclease includes a Cas9 nuclease (also known as Csn1 and Csx12), nickase, or a variant thereof.

In some aspects, the nuclease includes C2c1, C2c2, C2c3, Cas1, Cas1B, Cas2, Cas3, Cas4, Cas5, Cas6, Cas7, Cas8, Cas9, Cas10, Cpf1, Csy1, Csy2, Csy3, Cse1, Cse2, Csc1, Csc2, Csa5, Csn1, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx100, Csx16, CsaX, Csx3, Csx1, Csx15, Csx12, Csf1, Csf2, Csf3, Csf4, Csf1, Csf2, Csf3, Csf4, Cpf1, CasX, CasY, Argonaute, and any homologs or modified versions or similar nuclease, nickase, variants thereof, and/or combinations thereof.

As used herein, a “G-quadruplex” or “GQ” refers to structures adopted by both DNA and RNA, and is formed by the stacking of G-tetrad units and further stabilized by metal cations. In some aspects, GQ-forming sequences are identified by genome-wide computational studies and high-throughput sequencing. In some aspects, GQ-forming sequences comprise at least four segments of NGGN sequences. In some aspects, GQ-forming sequences comprise the nucleic acid sequence 5′-UAGGGUUAGGGU-3′. In some aspects, GQ-forming sequences comprise the nucleic acid sequence 5′-TAGGGTTAGGGT-3′. In some aspects, the GQ formed is a DNA/RNA parallel hetero-G-quadruplex. In some aspects, the GQ formed is a DNA/RNA antiparallel hetero-G-quadruplex. In some aspects, the GQ formed has mixed topology. In some aspects, the GQ is formed intermolecularly. In some aspects, the GQ is formed intramolecularly. In some aspects, the GQ-forming sequence is 3, 4, 5, 6, 7, 8, 9, 10, 11, 12, 13, 14, 15, 16, 17, 18, 19, or 20 nucleotides in length. In some aspects, the GQ-forming sequence is between 1 nucleotide and 10 nucleotides, between 5 nucleotides and 20 nucleotides, between 10 nucleotides and 50 nucleotides in length.

As used herein, a “heteroduplex barcode” refers to a barcode used to directly non-covalently link two nucleic acid molecules (e.g. sgRNA to donor DNA). In some aspects, the heteroduplex barcode sequence is 5, 10, 11, 12, 13, 14, 15, 16, 17, 18, 19, 20, 21, 22, 23, 24, 25, 30, or 35 nucleotides in length. In some aspects, the heteroduplex barcode sequence is between 1 nucleotide and 10 nucleotides, between 5 nucleotides and 20 nucleotides, between 10 nucleotides and 50 nucleotides in length. In some aspects, the heteroduplex barcode sequence is at least 1, at least 5, at least 10, at least 20, at least 30, at least 40, or at least 50 nucleotides in length.

The present disclosure relates to methods and compositions for suppressing non-homologous end joining (NHEJ) and/or favoring homology directed repair (HDR) to enable precision editing at a given genomic locus. The disclosure also relates to methods of using NHEJ or other non-HDR related pathways to incorporate genetic changes at a desired genomic site. HDR can be used to introduce precise edits by both introducing a DNA double-strand break or nick in the genome at a user-specified site and by introducing an exogenous piece of DNA (e.g., a repair template) with the desired edit(s), where the desired edit(s) in the repair template are flanked by regions of homology to the site of the double-strand break or single-strand nick. The repair template can be a single-stranded oligonucleotide donor DNA (ssODN) or a double-stranded donor DNA and can be contained on a linear or circular piece of DNA. CRISPR-mediated HDR, however, occurs with very low efficiency. The present methods and compositions present novel strategies for the direct recruitment of repair templates to the site of genomic lesion. The molecular “tethers” described herein result in an increase in the local concentration of repair templates at the lesion site, thereby enhancing the rate of HDR and suppressing undesired edits.

In one aspect, there is provided a fusion or noncovalent association between two nucleases (e.g., Cas proteins) and a third protein or moiety that recruits a donor to the site of nuclease-induced DNA damage. In one aspect, there is provided a method for increasing genome repair during CRISPR editing of genomes in a population of cells comprising the steps of: designing and synthesizing a library of editing cassettes wherein each of the editing cassettes encodes a recruitment construct comprising a gRNA, a repair template, and a binding moiety of a binding pair; designing and synthesizing a fusion enzyme construct comprising first and second orthogonal nucleases and a recruiting moiety of the binding pair; forming a ribonucleoprotein (RNP) complex with the recruitment construct and the fusion enzyme construct; introducing the RNP into cells to be edited; providing conditions for editing in the cells; and enriching for edited cells, wherein the recruiting moiety of the fusion enzyme construct binds to the binding moiety of the editing cassettes thereby bringing the editing cassettes and the fusion enzyme construct into proximity with one another. In some aspects, genome repair is made by homology directed repair (HDR), and in some aspects, the genome repair is made by non-homologous end joining (NHEJ) repair.

In some aspects, the recruiting moiety is streptavidin and the binding moiety is biotin or the recruiting moiety is biotin and the binding moiety is streptavidin. In some aspects, the recruiting moiety is Epstein-Barr virus (EBV)-encoded nuclear antigen-1 (EBNA1) and the binding moiety is an origin of plasmid replication (oriP) or the recruiting moiety is oriP and the binding moiety is EBNA1. EBNA1 binds to repetitive DNA recognition elements in the oriP. In some aspects, the recruiting moiety is SV40 T-antigen and the binding moiety is SV40 origin of replication or the recruiting moiety is SV40 origin of replication and the binding moiety is SV40 T-antigen; in some aspects the recruiting moiety is BK T-antigen and the binding moiety is BK Virus (BKV) origin of replication or the recruiting moiety is BKV origin of replication and the binding moiety is BK T-antigen; in some aspects, the recruiting moiety is latency-associated nuclear antigen (LANA1) of Karposi's Sarcoma Herpesvirus (KSHV) and the binding moiety is LANA binding site (LBS) of KSHV or the recruiting moiety is LANA binding site of KSHV and the binding moiety is LANA1 of KSHV; in some aspects, the recruiting moiety is minichromosome maintenance element (MME) region of human papilloma virus (HPV) and the binding moiety is E2 protein of HPV; in some aspects, the recruiting moiety is an HUH-tag and the binding moiety is an HUH recognition sequence or the recruiting moiety is an HUH recognition sequence and the binding moiety is an HUH-tag; in some aspects, the recruiting moiety is a Udg variant and the binding moiety is uracilated DNA or the recruiting moiety is uracilated DNA and the binding moiety is a Udg variant; and in some aspects, the recruiting moiety is a retron and the binding moiety is retron-synthesized RNA.

In some aspects, the recruiting moiety of the binding pair is positioned between the first and second orthogonal nucleases, and in some aspects, the recruiting moiety of the binding pair is positioned N-terminal to the first and second orthogonal nucleases, and in yet some aspects, the recruiting moiety of the binding pair is positioned C-terminal to the first and second orthogonal nucleases.

In some aspects, the first and second orthogonal nucleases are Type II nucleases; in some aspects, the first orthogonal nuclease is Streptococcus pyogenes Cas9 (SpCas9) and the second orthogonal nuclease is Staphylococcus aureus Cas9 (SaCas9). In some aspects the first and second orthogonal nucleases are Type V nucleases. In some aspects, the first orthogonal nuclease is a Type II nuclease and the second orthogonal nuclease is a Type V nuclease. In some aspects, one of the nucleases is a dead nuclease.

### Nucleic Acid-Guided Nickase/Reverse Transcriptase Fusion Enzyme Genome Editing Generally

The compositions and methods described herein are a “twist on” or alternative to traditional nucleic acid-guided nuclease editing (e.g., RNA-guided nuclease editing or CRISPR editing) used to introduce desired edits to a population of cells; that is, the compositions and methods described herein employ a fusion enzyme construct—as opposed to a CRISPR nuclease—and an editing cassette comprising a binding moiety—as opposed to an editing cassette—to increase HDR, which thereby increases precision editing in a cell population. A nucleic acid-guided nuclease complexed with an appropriate synthetic guide nucleic acid in a cell can cut the genome of the cell at a desired location. The guide nucleic acid helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects, the guide nucleic acid is a single guide nucleic acid construct that includes both (1) a guide sequence capable of hybridizing to a genomic target locus, and (2) a scaffold sequence capable of interacting or complexing with a nucleic acid-guided nuclease enzyme.

In general, a guide nucleic acid (e.g., gRNA) complexes with a compatible nucleic acid-guided nuclease and can then hybridize with a target sequence, thereby directing the nuclease to the target sequence. A guide nucleic acid can be DNA or RNA; alternatively, a guide nucleic acid may comprise both DNA and RNA. In some aspects, a guide nucleic acid may comprise modified or non-naturally occurring nucleotides. In some aspects, a guide nucleic acid comprises RNA, and the gRNA may be encoded by a DNA sequence on a polynucleotide molecule such as a plasmid, linear construct, or the coding sequence may and preferably does reside within a source editing cassette. Methods and compositions for designing and synthesizing editing cassettes are described in U.S. Pat. Nos. 9,982,278; 10,266,849; 10,240,167; 10,351,877; 10,364,442; 10,435,715; 10,465,207; 10,669,559; 10,771,284; 10,731,498; and 11,078,498.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 55%, 60%, 65%, 70%, 75%, 80%, 85%, 90%, 92.5%, 95%, 97.5%, 99%, or more. Optimal alignment may be determined with the use of any suitable algorithm for aligning sequences (e.g., without being limiting, BLAST™). In some aspects, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some aspects, a guide sequence is less than about 75, 50, 45, 40, 35, 30, 25, 20 nucleotides in length. Preferably the guide sequence is between 10 nucleotides and 30 nucleotides long, between 15 nucleotides and 20 nucleotides long, or 15, 16, 17, 18, 19, or 20 nucleotides in length.

In general, to generate an edit in the target sequence, the gRNA/nuclease complex binds to a target sequence as determined by the guide RNA, and the nuclease recognizes a protospacer adjacent motif (PAM) sequence adjacent to the target sequence. The target sequence that is recognized and bound by the gRNA/nuclease complex can be any polynucleotide endogenous or exogenous to the cell, or in vitro. For example, the target sequence can be a polynucleotide residing in the nucleus of the cell. A target sequence can be a sequence encoding a gene product (e.g., a protein) or a non-coding sequence (e.g., a regulatory polynucleotide, an intron, a PAM, a control sequence, or “junk” DNA).

The guide nucleic acid may be and preferably is part of an editing cassette that encodes the repair template that targets a cellular target sequence. Alternatively, the guide nucleic acid may not be part of the editing cassette and instead may be encoded on the editing vector backbone or as a linear piece of DNA. For example, a sequence coding for a guide nucleic acid can be assembled or inserted into a vector backbone first, followed by insertion of the repair template in, e.g., an editing cassette. In other aspects, the repair template in, e.g., an editing cassette can be inserted or assembled into a vector backbone first, followed by insertion of the sequence coding for the guide nucleic acid. In an aspect, the nucleic acid sequence encoding the guide nucleic acid and the repair template are located together in a rationally-designed editing cassette. In an aspect, the nucleic acid sequence encoding the guide nucleic acid and the repair template are located apart in a rationally-designed editing cassette. The target sequence is associated with a proto-spacer adjacent motif (PAM), which is a short nucleotide sequence recognized by the gRNA/nuclease complex. The precise preferred PAM sequence and length requirements for different nucleic acid-guided nucleases vary; however, PAMs typically are 2-7 base-pair sequences adjacent or in proximity to the target sequence and, depending on the nuclease, can be 5′ or 3′ to the target sequence. Engineering of the PAM-interacting domain of a nucleic acid-guided nuclease may allow for alteration of PAM specificity, improve target site recognition fidelity, decrease target site recognition fidelity, or increase the versatility of a nucleic acid-guided nuclease.

In most aspects, genome editing of a cellular target sequence both introduces a desired DNA change to a cellular target sequence (an “intended” edit), e.g., the genomic DNA of a cell, and removes, mutates, or renders inactive a PAM or spacer region in the cellular target sequence (an “immunizing edit”) thereby rendering the target site immune to further nuclease binding. Rendering the PAM at the cellular target sequence inactive precludes additional editing of the cell genome at that cellular target sequence, e.g., upon subsequent exposure to a nucleic acid-guided nuclease complexed with a synthetic guide nucleic acid in later rounds of editing. As for the nuclease component of the nucleic acid-guided nuclease editing system, a polynucleotide sequence encoding the nucleic acid-guided nuclease can be codon optimized for expression in particular cell types, such as bacterial, yeast, and mammalian cells. The choice of the nucleic acid-guided nuclease to be employed depends on many factors, such as what type of edit is to be made in the target sequence and whether an appropriate PAM is located close to the desired target sequence. Nucleases of use in the methods described herein include but are not limited to Cas 9, Cas 12/Cpf1, MAD2, MAD7®, or other MADZYME®. If a nickase is used instead of a nuclease, the nickase may be developed or derived from, e.g., Cas 9, Cas 12/Cpf1, MAD2, or MAD7 or other MADzymes. For more information on MADzymes, please see U.S. Pat. Nos. 10,604,746; 10,655,114; 10,649,754; 10,876,102; 10,833,077; 11,053,485; 10,704,022; 10,745,678; 10,724,021; 10,767,169; 10,870,761; 10,011,849; 10,435,714; 10,626,416; 9,982,279; and 10,337,028; and U.S. Ser. Nos. 16/953,253; 17/374,628; 17,200,074; 17,200,089; 17/200,110; 16/953,233; 17/463,498; 63/134,938; 16/819,896; 17/179,193; and Ser. No. 16/421,783.

Another component of the nucleic acid-guided nuclease system is the repair template comprising homology to the cellular target sequence. For the present methods and compositions, the repair template is preferably in the same editing cassette as the guide nucleic acid. The repair template is designed to serve as a template for homologous recombination with a cellular target sequence cleaved or nicked by the fusion enzyme construct as a part of the gRNA/nuclease nickase fusion enzyme complex or as a donor that may be incorporated via NHEJ and without homology. A repair template polynucleotide may be of any suitable length, such as about or more than about 20, 25, 50, 75, 100, 150, 200, 500, or 1000 nucleotides in length, and up to 2, 3, 4, 5, 6, 7, 8, 9, 10, 11, 12, 13 and up to 20 kb in length. In certain preferred aspects, the repair template can be provided as an oligonucleotide of between 20 nucleotides and 300 nucleotides in length, more preferably between 50 nucleotides and 250 nucleotides in length. The repair template comprises regions that are complementary to a portion of the cellular target sequence (e.g., homology arms). When optimally aligned, the repair template overlaps with (is complementary to) the cellular target sequence by, e.g., about 20, 25, 30, 35, 40, 50, 60, 70, 80, 90 or more nucleotides. In some aspects, the two homology arms (regions complementary to the cellular target sequence) flank the mutation or difference between the repair template and the cellular target sequence. In some aspects, the repair template comprises at least one mutation or alteration compared to the cellular target sequence, such as an insertion, deletion, modification, or any combination thereof compared to the cellular target sequence.

In some aspects, the repair template is delivered to the cells as part of a ribonucleoprotein (RNP) complex. In some aspects, the repair template is provided as part of a rationally-designed editing cassette, which can be inserted into an editing plasmid backbone where the editing plasmid backbone may comprise a promoter to drive transcription of the editing gRNA and the repair template when the editing cassette is inserted into the editing plasmid backbone; however in some aspects, there is a 5′ or 3′ handle or internal modification that enables the protein to bind to the repair template which may preclude insertion of the editing cassette into a vector. In aspects where the editing cassette is inserted into a vector, the promoter driving transcription of the editing gRNA and the repair template (or driving more than one editing gRNA/repair template pair) is optionally an inducible promoter.

In addition to the repair template, an editing cassette may comprise one or more primer binding sites. The primer binding sites are used to amplify the editing cassette by using oligonucleotide primers as described infra and may be biotinylated or otherwise labeled. In some aspects, the editing cassettes comprise a collection or library of editing gRNAs and of repair templates representing, e.g., gene-wide or genome-wide libraries of editing gRNAs and repair templates. The library of editing cassettes may also include a barcode, where, each different repair template is associated with a different barcode.

### EXAMPLES

### Example 1: Direct Recruitment of Repair Templates to CRISPR Fusion Enzyme Constructs to Increase HDR

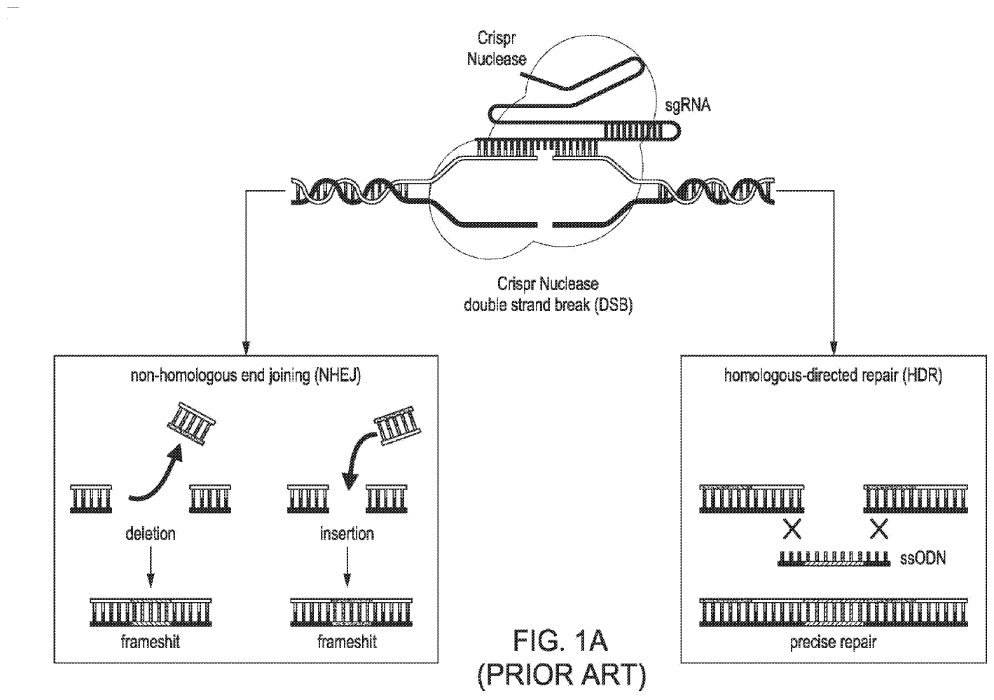

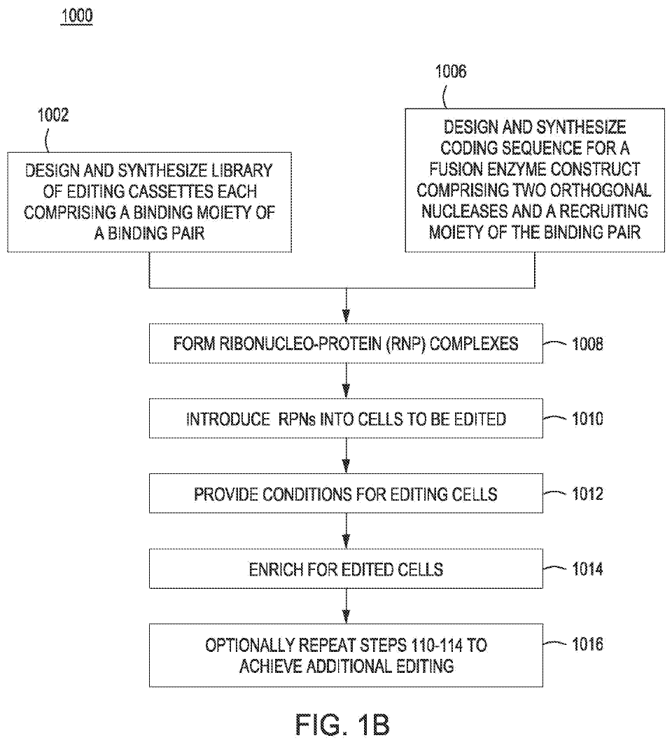

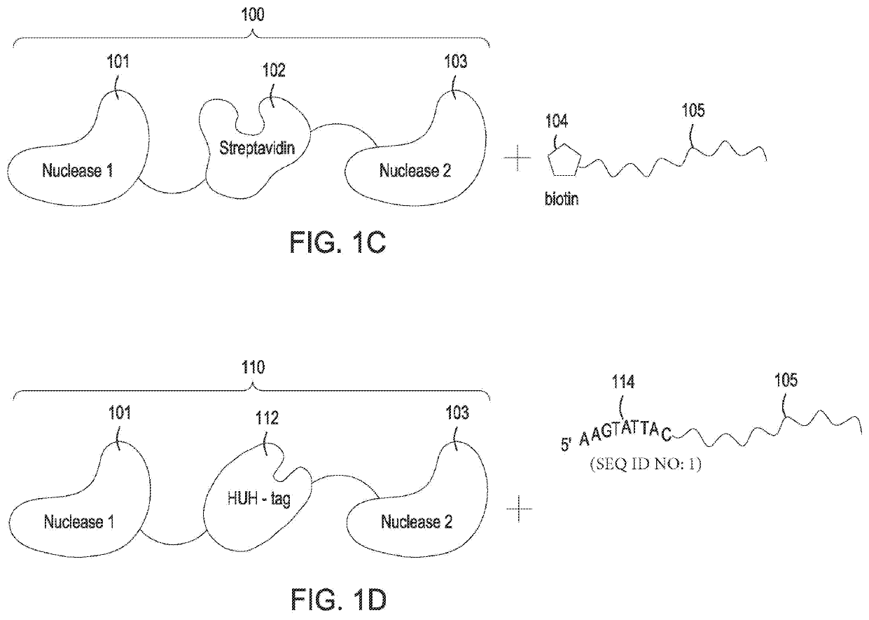

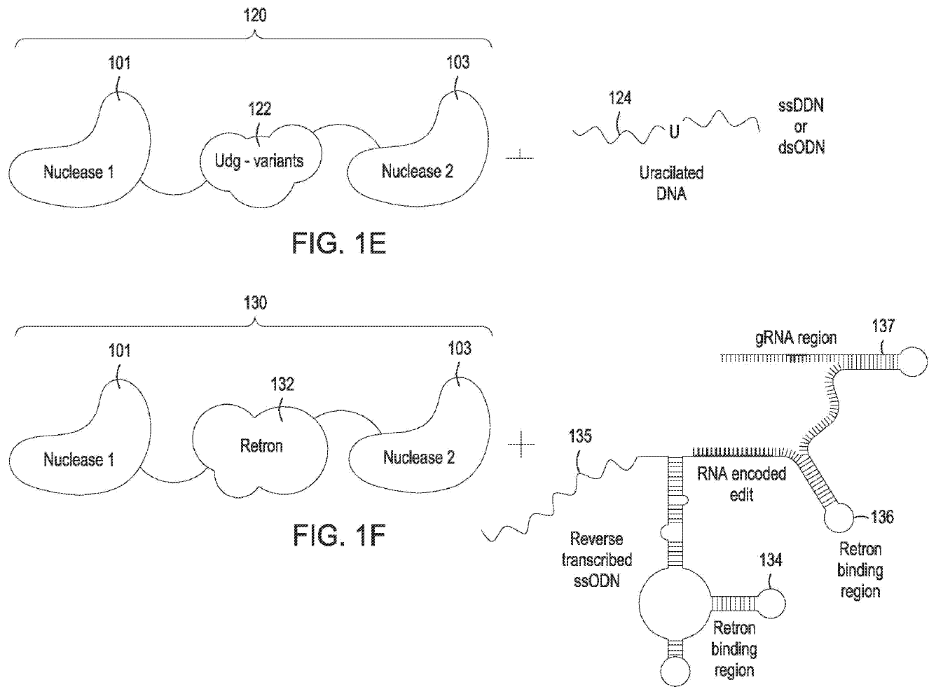

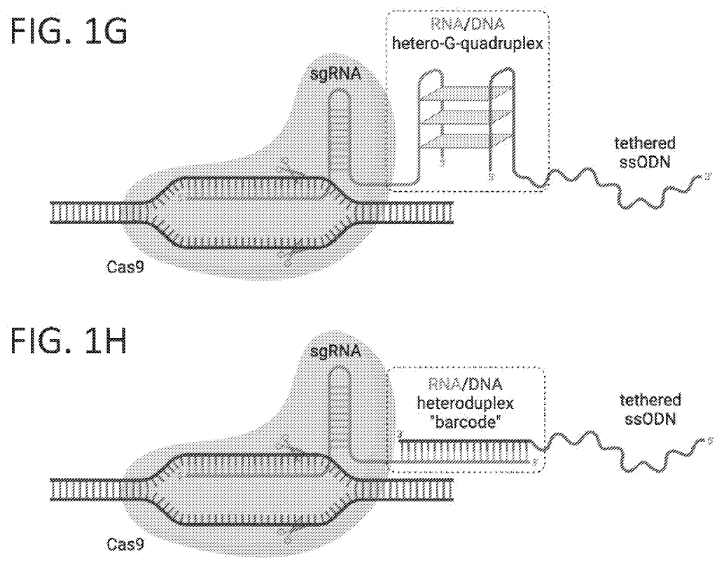

The present disclosure provides compositions of matter, methods and instruments for directly recruiting repair templates to CRISPR nucleases to stimulate homology-directed repair (HDR). HDR is a process whereby a double-strand break (DSB) is repaired using a second DNA strand homologous to an area surrounding the break, a repair mechanism which generally utilizes the replicated sister chromatid. However, HDR can be used to introduce precise edits by introducing a DNA DSB or nick in the genome at a user-specified site and by introducing an exogenous piece of DNA with the desired edit, flanked by regions of homology (donor) to the break-site. A repair template can be a single-stranded oligonucleotide donor DNA (ssODN) or a double-stranded donor DNA and can be contained on a linear or circular piece of DNA. CRISPR-mediated HDR, however, occurs with very low efficiency. The present disclosure describes strategies for the direct recruitment of a repair template to the site of genomic lesion. The molecular “tethers” described here result in an increase in the local concentration of repair template at the double-stranded break (i.e., lesion) site, thereby enhancing the rate of HDR and suppressing undesired edits. Another mechanism, non-homologous end joining (NHEJ), repairs double-strand breaks induced by CRISPR nucleases via repair and/or protection of the break site and re-ligation. NHEJ is known to incorporate double-stranded fragments of DNA and has been used to integrate linear fragments of DNA in a method known as Homology Independent Targeted Integration. FIG. 1A shows an overview of the DNA repair pathways—HDR and NHEJ—following CRISPR nuclease-induced double-strand breaks. The panel at right in FIG. TA shows NHEJ and at left shows HDR. Some methods have been described to recruit DNA donor sequences to target sites by tethering DNA donor sequences to a gRNA-Cas9 complex comprising a single nuclease (See Zhang et al., “Homology-based repair induced by CRISPR-Cas nucleases in mammalian embryo genome editing,” Protein Cell, May 2022, 13(5):316-335, e-published May 4, 2021). However, the compositions and methods of the present disclosure solve two key issues in CRISPR gene editing using homology directed repair: (1) direct attachment of the repair template to the Cas9 ribonucleoprotein complex; and (2) increase of usable PAM sites. Repair template availability is solved by forcing the recruitment construct to the site of the double-stranded beak via a binding pair comprised by the recruitment construct and a recruiting moiety coupled to two orthogonal nucleases in a fusion enzyme construct. The PAM requirements for a given single CRISPR nuclease are relaxed by fusion of two orthogonal CRISPR nucleases in the fusion enzyme construct. Such dual nuclease platforms not only enable various lesion types (e.g. double-strand breaks, single- or double-strand nicks, etc.) but also may enable a higher frequency of on-target cleavage when the edit site is specified by more than one nuclease; e.g. SpCas9^{WT} (Streptococcus pyogenes Cas9) and SaCas9^{dead} (Staphylococcus aureus Cas9) with two guide RNAs. A reliance on two gRNA target sites, each creating a nick for example, is an established method for enabling DSB-mediated repair while also decreasing the individual mutagenic off-target effects of any single gRNA. Because nicks are generally less mutagenic than DSBs, a single off-target event is less likely to cause unwanted mutations relative the DSB caused by the off-target cleavage event of a single gRNA Cas9 complex. The present compositions and methods provide for the PAM relaxation benefits of dual nuclease platforms with the enhancement of HDR using a genetic fusion of three proteins: two orthogonal nucleases which are further fused to a recruiting moiety.

There are several novel and unusual features of the compositions and methods of the present disclosure. First, dual nuclease fusions have been used previously to generate programmed deletions (see, e.g., Bolukbasi, et al., Nature Corn. 9:4856(2018)) but have not been used for repair template-mediated precise repair. Second, the present methods allow for efficient incorporation of longer stretches of DNA than, e.g., prime editing methods (see, e.g., Anazalone, et al., Nature 576:149-157 (2019)); thus, enabling genome-wide insertions of recombinase sites, protein degron tags, promoters, terminators, alternative-splice sites, and CpG islands. Third, the present methods allow for long insertions or deletions of, e.g., introns, exons, repetitive elements, promoters terminators insulators, CpG islands, non-coding elements, retrotransposons, and retroviruses. Fourth, the present methods allow for increased accessibility of previously inaccessible genomic regions due to the increased PAM site recognition of the dual orthogonal nucleases.

FIG. 1B is a simplified block diagram of an example of method 1000 for editing live cells via fusion enzyme construct editing. Looking at FIG. 1B, method 1000 begins by designing and synthesizing a library of editing cassettes wherein each editing cassette comprises a gRNA, a repair template, and a binding moiety 1002 (e.g., an “editing cassette with a binding moiety” or “ECBM”), where the repair template comprises the desired target genome edit(s) as well as a PAM or spacer mutation. In addition, a fusion enzyme construct is designed and synthesized 1006. In many aspects, once the ECBMs and the fusion enzyme constructs are synthesized 1002, 1006, they are combined to form ribonucleoprotein (RNP) complexes 1008. At step 1010, the RNPs are introduced into the live cells.

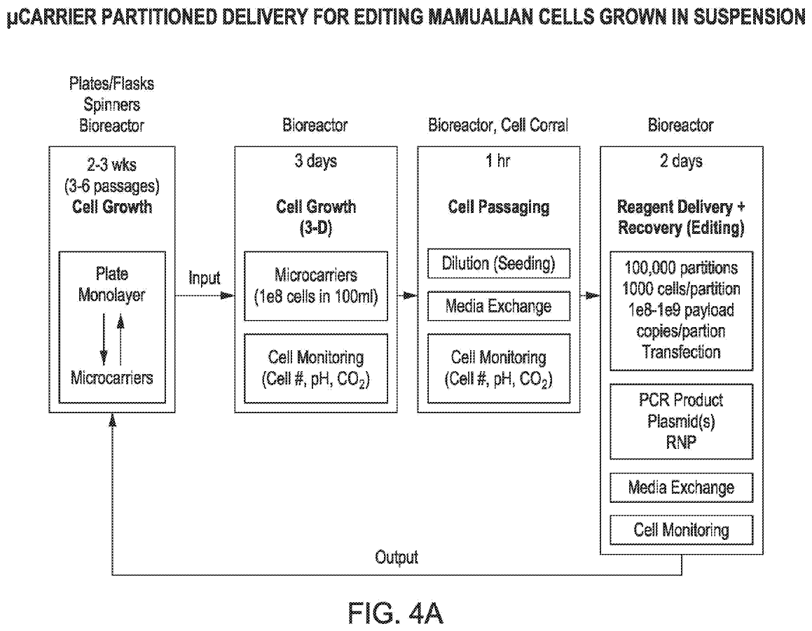

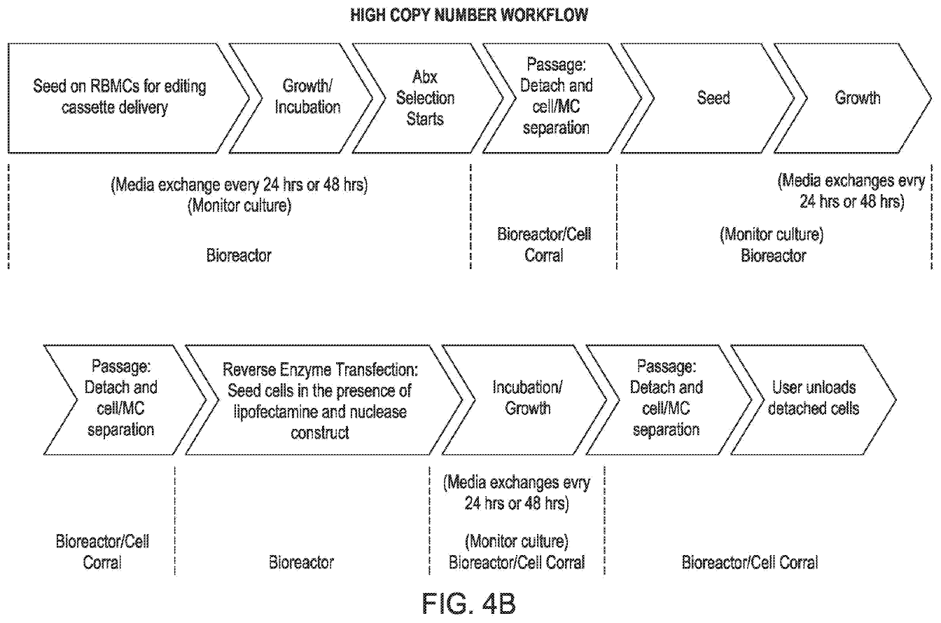

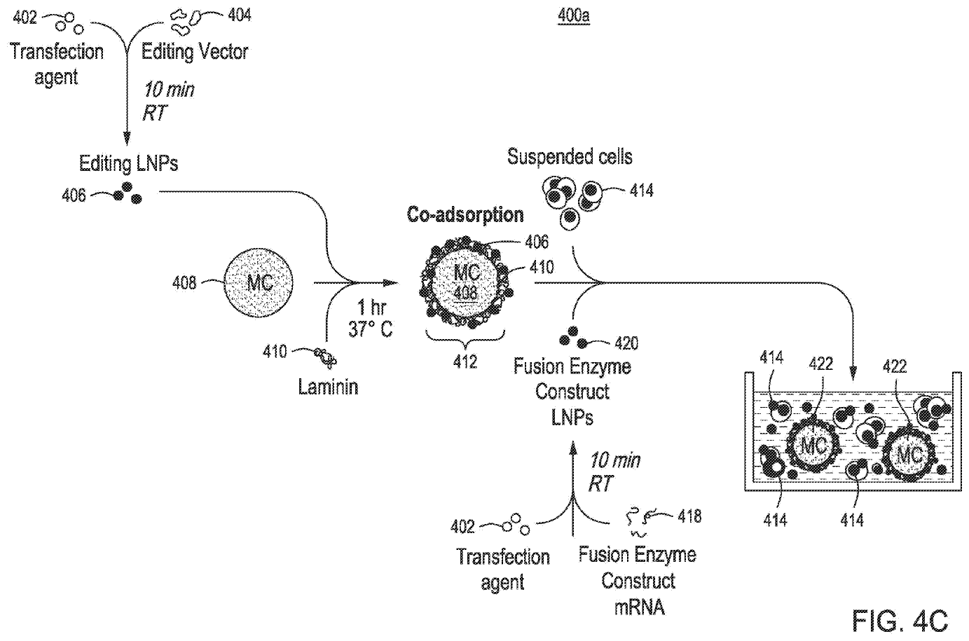

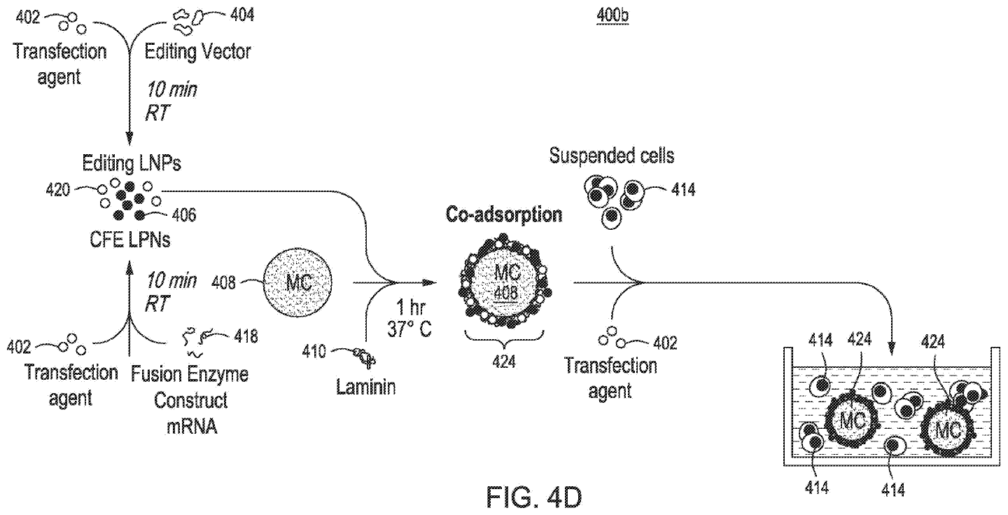

A variety of delivery systems may be used to introduce (e.g., transform or transfect) the fusion enzyme editing components into a host cell 1008. These delivery systems include the use of yeast systems, lipofection systems, microinjection systems, biolistic systems, virosomes, liposomes, immunoliposomes, polycations, lipid:nucleic acid conjugates, virions, artificial virions, viral vectors, electroporation, cell permeable peptides, nanoparticles, nanowires, exosomes. Alternatively, molecular trojan horse liposomes may be used to deliver nucleic acid-guided nuclease components across the blood brain barrier. Of particular interest is the use of electroporation, particularly flow-through electroporation (either as a stand-alone instrument or as a module in an automated multi-module system) as described in. e.g., U.S. Pat. Nos. 10,253,316; 10,329,559; 10,323,242; 10,421,959; 10,465,185; 10,519,437; and U.S. Ser. Nos. 16/666,964, filed 29 Oct. 2019, and Ser. No. 16/680,643, filed 12 Nov. 2019; and microcarrier-based transfection as described in FIGS. 4C and 4D and the text pertinent thereto infra, as well as described in U.S. Ser. No. 17/239,538, filed 23 Apr. 2021 and Ser. No. 17/239,540, filed 23 Apr. 2021.

Once transformed 1010, the next step in method 1000 is to provide conditions for fusion enzyme construct editing 1012. “Providing conditions” includes incubation of the cells in appropriate medium and may also include providing conditions to induce transcription of an inducible promoter (e.g., adding antibiotics, increasing temperature) for transcription of one or both of the ECBMs and the fusion enzyme construct. Once editing is complete, the cells are allowed to recover and are preferably enriched for cells that have edited 1014 or have received a co-delivered selectable marker. Enrichment can be performed directly, such as via cells from the population that express a selectable marker, or by using surrogates, e.g., cell surface handles co-introduced with one or more components of the editing components and using cell sorting, e.g., using FACs (fluorescent activated cell sorting). At this point in method 1000, the cells can be characterized phenotypically or genotypically or optionally steps 1010-1014 may be repeated to make additional edits 1016.

### Example 2: Dual Nuclease-Streptavidin Fusion

One aspect of the present CRISPR editing systems employing a fusion enzyme construct comprising orthogonal nucleases and a recruiting moiety and an ECBM is shown in FIG. 1C. This aspect combines two concepts and their attendant benefits: (i) the genetic fusion of orthogonal nucleases to form a dual nuclease construct, and (ii) the recruitment of the repair template to the fusion enzyme construct where the recruitment moiety is a streptavidin protein and the repair template is biotinylated. Dual/orthogonal nuclease platforms have been shown to relax protospacer adjacent motif (PAM) requirements for putative cleavage sites in the genome; for instance, whereas SpCas9 (S. pyogenes Cas9) has a strong preference for NGG PAMs, a SpCas9-NmeCas9 (N. meningitides Cas9) fusion protein is active at NGN and NNG PAMs when both orthogonal guide RNAs are expressed (see e.g., Bolukbasi, et al., Nature Corn. 9:4856(2018) describing the Cas9-Cas9 fusions). In some aspects, the Staphylococcus aureus Cas9 (SaCas9) used is an evolved, modified “KKH” variant that recognizes NNNRRT PAM sequences (SEQ ID NO: 4), rather than the canonical NNGRRT PAM sequences (SEQ ID NO: 5).

Separately, an RNA aptamer for streptavidin (termed S1m) has been incorporated into the canonical guide RNA scaffold, and this fused guide RNA has been shown to form an active ribonucleoprotein (RNP) cleavage complex with SpCas9. Upon further complexation with streptavidin (SA) and biotinylated ssODN (now termed “S1mplex”), homology-directed repair was enhanced. (For additional information on use of S1mplex to enhance precise genome editing via HDR see, e.g., Carlson-Stervermer, et al., Nature Communications, 8:1711, DOI: 10.1038/s41467-017-01875-9 (2017).) Cas9 has also been directly fused with streptavidin, enabling recruitment of a donor and upregulation of HDR. (See, e.g., Ma, et al., Cell Research, 27:578-81 (2017); and Pineault, et al., MethodsX, 6:2088-2100 (2019).) The present strategy combines the PAM relaxation benefits of dual/orthogonal nuclease platforms with the enhancement of HDR by using the genetic fusion of three proteins: two orthogonal nucleases which are further fused to—in this aspect—(monomeric) streptavidin.

The dual nucleases—orthogonal nucleases 101, 103—used in the fusion enzyme construct 100 may be any Cas nucleases where the gRNAs are orthogonal; for example, SpCas9 gRNA and SaCas9 (S. aureus Cas9) gRNA are known to not cross-react. Type II nucleases and Type V nucleases may be used, as long as the gRNAs of the nucleases in the fusion enzyme construct are orthogonal. In addition to use of orthogonal CRISPR nucleases, the nucleases can be replaced with nickase variants and the mechanism would still broadly be classified as “homology directed repair”, as the ssODN repair template is still directing a precise edit, regardless of the lesion or type of break or nick induced by the nuclease, nickase or variants. That is, in the cell the actual mechanism may vary depending on the type of lesion, but the outcome/readout would be the same.

As for the binding moiety/recruitment moiety binding pair, in this aspect, biotin 104 and streptavidin 102 are shown; however, other binding moiety/recruiting moiety pairs may be employed such as EBNA1 and oriP; SV40 T-antigen and SV40 origin of replication; BK T-antigen and BKV origin of replication; LANA1 (latency-associated nuclear antigen) and LANA binding site (LBS) of KSHV (Karposi's Sarcoma Herpesvirus); and E2 protein and MME region (minichromosome maintenance element) of HPV (human papilloma virus), as well as the other binding moiety/recruitment moiety binding pairs exemplified below in FIGS. 1D-1F. Additional sequence or structure specific binding proteins such as transcription factors, may also be used to recruit proteins may also be used to recruit donor DNA. Note that the binding moiety is coupled with the gRNA and repair template 105 in the editing cassette. The binding moiety will depend on the moiety/recruitment moiety used. For example, HUH domains and their cognate recognition sites must be present on the 5′ end of a single stranded DNA; the covalent bond formation results in cleavage and removal of the 5′ end of DNA. In contrast, uracilated deoxyribonucleic acid can be incorporated within the DNA strand for recognition by UdgX. The gRNA, if fused to the donor, may therefore be linked at the 5′ or 3′ end of the donor, depending on the particular binding moiety/recruitment moiety used.

Additionally, in the fusion enzyme construct 100 shown in FIG. 1C (and those in FIGS. 1D-1F), the recruitment moiety 102 is shown as being positioned between the orthogonal nucleases; however, there is no a priori limitation on the “order” of the protein domains; thus, any one of nuclease 1 101, nuclease 2 103 and the recruitment moiety 102 can be positioned at the N-terminus of the fusion enzyme construct; any one of the nuclease 1 101, nuclease 2 103 and the recruitment moiety 102 can be positioned in the middle of the fusion enzyme construct; and any one of the nuclease 1 101, nuclease 2 103 and the recruitment moiety 102 can be positioned at the C-terminus of the fusion enzyme construct.

### Example 3: Dual Nuclease-PCV Fusion

A second example of the presently described CRISPR editing systems employing a fusion enzyme construct comprising orthogonal nucleases and a recruiting moiety and an ECBM is shown in FIG. 1D. This example combines the dual orthogonal nucleases with another class of ssDNA binding protein: HUH endonucleases (or HUH-tags). HUH endonucleases are sequence-specific single-stranded DNA (ssDNA) binding proteins originating from numerous species of bacteria and viruses. HUH endonucleases can be used to create protein-DNA linkages, and in doing so a 5′ covalent bond is created between the ssDNA and the protein. HUH endonucleases can be fused with other proteins or used as protein tags and are broadly split into two categories of enzymes: replication initiator proteins (Rep) or relaxase/mobilization proteins. For example, the Rep protein derived from porcine circovirus 2 (PCV2) recognizes a short 9-mer sequence on ssDNA (5′-AAGTATTAC-3′) (SEQ ID NO: 1) and liberates the terminal 2-mer to generate a covalent bond between the PCV2 protein and ssDNA. Previously, a Cas9-PCV2 fusion protein has been shown to increase HDR rates when combined with a ssODN that included a 5′-terminal PCV recognition sequence. (For more information, see Aird, et al., Communications Biology, DOI:101038/242003-018-0054-2 (2018).) Like the CRISPR editing system described above, the dual nuclease 101, 103 PCV approach swaps in a HUH-tag 112 such as PCV2 in lieu of streptavidin as a recruitment moiety 112 for the binding moiety 114 (e.g., an HU recognition sequence) coupled to the editing cassette 105 comprising the gRNA and repair template. That is, here, the orthogonal nucleases nuclease 1 101 and nuclease 2 103 are fused to a HUH-tag 112 and combined with a ECBM that comprises the editing cassette 105 HUH recognition sequence 114. The covalent complex between the fusion enzyme construct 110 and the ECBM 105+114 increases the rates of HDR via recruitment of the donor template directly to the break site. In this aspect as with the aspect shown in FIG. 1C and described above, dual orthogonal nickases can be used in lieu of dual orthogonal nucleases and the order of the nuclease 1, nuclease 2 and the recruiting moiety (here, the HUH recognition sequence) may be varied.

### Example 4: Dual Nuclease-Udg and UdgX Fusion

A third example of the presently described CRISPR editing systems employing a fusion enzyme construct comprising orthogonal nucleases and a recruiting moiety and an ECBM is shown in FIG. 1E. Uracil deglycosylases recognize and remove uracil from DNA. Unlike most other uracil DNA glycosylases, however, UdgX results in excision of uracil followed by covalent attachment to DNA at the newly created abasic site. Additionally, a number of mutant uracil DNA glycosylases could be used that form strong, noncovalent interactions with uracilated DNA. Similar to the methods above, fusion of UdgX 122 to the dual orthogonal nucleases nuclease 1 101, nuclease 2 103 in the fusion enzyme construct 120 combined with a uracilated editing cassette (ECBM) 124 tethers the ECEB 124 to the fusion enzyme construct 120. Donor recruitment to the site of a Cas9 or dual Cas9 caused break enables can then enable upregulated HDR as previously described.

In this aspect, the repair template is fused away from the 5′ and 3′ termini of the donor DNA, allowing for both DNA ends to be available for enzymatic chemistry. Covalently tethering the repair template without homology may enable NHEJ-mediated insertion of the exogenous DNA; because the UdgX protein is coupled to two nuclease orthologs (nuclease 1 and nuclease 2), the sequence between the cut sites may then be removed and replaced with the repair template.

In addition to UdgX, uracil DNA glycosylases and variants are known to form strong, non-covalent interactions with the uracilated DNA, for more information see Slupphaug et al. Nature, 384(7):87-92 (1996) and Krusong et al. The Journal of Biological Chemistry, 241(8): 4983:4992 (2006). Fusion enzyme constructs comprising these other glycosylases are also advantageous and enable recruitment to the site of a DSB break, followed by slower disassembly after repair template incorporation. In this aspect as with the aspects shown in FIGS. 1C and 1D and described above, dual orthogonal nickases can be used in lieu of dual orthogonal nucleases and the order of the nuclease 1, nuclease 2 and the recruiting moiety (here, the UdgX) may be varied.

### Example 5: Dual Nuclease-Retron Fusion

A fourth example of the presently described CRISPR editing systems employing a fusion enzyme construct comprising orthogonal nucleases and a recruiting moiety and an ECBM is shown in FIG. 1F. Retrons are viral-like elements that reverse transcribe DNA from RNA. Upon reverse transcription, the newly synthesized DNA is covalently attached to the 3′ end of the RNA. Previous work has shown that fusing retro-elements to Cas9, co-transfected with a retro-element-gRNA fusion, can result in upregulation of HDR in mammalian cells. The retron contains invariant RNA sequences that can flank the 5′ and 3′ regions of the edit encoding donor. The invariant regions enable the retron's reverse transcriptase to reverse transcribe the intervening RNA sequence into single stranded DNA. This retron donor RNA can be fused to the gRNA. Cotransfection with a Cas9-retron or Cas9-Cas9-retron protein and this modified gRNA-retron-donor RNA enables the Cas9 to cleave at a desired site, and reverse transcribe a donor. The reverse transcribed donor is subsequently attached to the 3′ end of the gRNA-retron-donor RNA which is tightly complexed to Cas9-retron or Cas9-Cas9-retron fusion, serving to localize the donor to the site of a DNA break. (For additional information on use of retrons to enhance precise genome editing via HDR see, e.g., Kong, et al., Protein & Cell, 12(11):899-902 (2021).) Like the aspects described supra, the repair template, once synthesized, will be tethered via the ECBM to the fusion enzyme construct. This occurs because the newly synthesized cDNA is covalently attached to the gRNA-retron fusion, which is tightly complexed to the nuclease. The advantage of this aspect is that repair template does not need to be supplied exogenously and can be fused directly to the encoded gRNA. Also, in this aspect as with the aspects shown in FIGS. 1C-1E and described above, dual orthogonal nickases can be used in lieu of dual orthogonal nucleases and the order of the nuclease 1, nuclease 2 and the recruiting moiety (here, the fusion enzyme construct itself) may be varied.

### Example 6: Donor DNA-sgRNA Hybridization—G-Quadruplex

In an aspect, donor DNA is directly hybridized to the single guide RNA (sgRNA) to form a G-quadruplex (GQ) structure that is further complexed with a nuclease, and the resulting ribonucleoprotein (RNP) cleavage complex is directed to the site of lesion. In some aspects, guide RNAs may be extended or modified at the 3′ end. In some aspects, guide RNAs may be extended or modified at the 5′ end. In some aspects, various single guide RNAs (sgRNAs) and single-stranded oligonucleotide donor DNA (ssODN) pools may be multiplexed.

An example of using GQ structures to non-covalently link sgRNA and ssODN for recruitment of donor DNA to the site of lesion to enhance precise genome editing via HDR is shown in FIG. 1G. In some aspects, addition of the GQ-forming sequence (5′-UAGGGUUAGGGU-3′) (SEQ ID NO: 2) to the 3′ end of the sgRNA spontaneously anneals to a corresponding sequence appended to either end of the ssODN (5′-TAGGGTTAGGGT-3′) (SEQ ID NO: 3) and forms a DNA/RNA parallel hetero-G-quadruplex. In some aspects, formation of a DNA/RNA parallel hetero-G-quadruplex from sgRNA and ssODN improves the half-life of the sgRNA in vivo. In some aspects, there are no chemical modifications of either the sgRNA or the ssODN that forms a DNA/RNA parallel hetero-G-quadruplex. In some aspects, there are chemical modifications of either the sgRNA or the ssODN that forms a DNA/RNA parallel hetero-G-quadruplex. In some aspects, DNA/RNA parallel hetero-G-quadruplex formed from sgRNA and ssODN are complexed with nuclease (e.g. Cas9) to form RNPs.

In some aspects, the approach of using GQ structures to non-covalently link the sgRNA and ssODN for recruitment of donor DNA to the site of lesion can be used in combination with any of the previously described methods of employing a fusion enzyme construct comprising orthogonal nucleases and a recruiting moiety and an ECBM (FIG. 1B-FIG. 1F).

### Example 7: Donor DNA-sgRNA Hybridization Example—Heteroduplex Barcode

In an aspect, donor DNA is directly hybridized to the sgRNA using a heteroduplex barcode, and the DNA/RNA heteroduplex is complexed with a nuclease resulting in an RNP cleavage complex that is directed to the site of lesion (shown in FIG. 1H). In an aspect, a short heteroduplex barcode sequence is added to the 3′ end of the sgRNA, and the reverse complement of that heteroduplex barcode sequence is added to either end of the ssODN donor. In an aspect, a short heteroduplex barcode sequence is added to the 5′ end of the sgRNA, and the reverse complement of that heteroduplex barcode sequence is added to either end of the ssODN donor. In some aspects, heating and annealing cycles will generate non-covalent sgRNA-ssODN hybrids from the heteroduplex barcoded sgRNA and ssODN (e.g. sgRNA-barcode:rev.comp, barcode-ssODN). In some aspects, non-covalent sgRNA-ssODN hybrids are complexed with nuclease (e.g. Cas9) to form RNPs. In some aspects, formation of sgRNA-ssODN hybrids improves the half-life of the sgRNA in vivo. In an aspect, nuclease-sgRNA-ssODN complexes formed by RNA/DNA heteroduplex barcoding hybridization will have no chemical modification of either the nuclease or the ssODN, or both. In an aspect, nuclease-sgRNA-ssODN complexes formed by RNA/DNA heteroduplex barcoding hybridization will have chemical modifications of either the nuclease or the ssODN, or both. In some aspects, unique heteroduplex barcode sequences allow for complex mixtures of sgRNAs targeting various genomic loci to hybridize only to their cognate ssODN.

In some aspects, the approach of using heteroduplex barcoding hybridization to non-covalently link the sgRNA and ssODN for recruitment of donor DNA to the site of lesion can be used in combination with any of the previously described methods of employing a fusion enzyme construct comprising orthogonal nucleases and a recruiting moiety and an ECBM (FIG. 1B-FIG. 1F).

### Example 8: Automated Cell Editing Instruments and Modules to Perform Nucleic Acid-Guided Nickase Fusion Editing in Cells

### One Example of an Automated Cell Editing Instrument

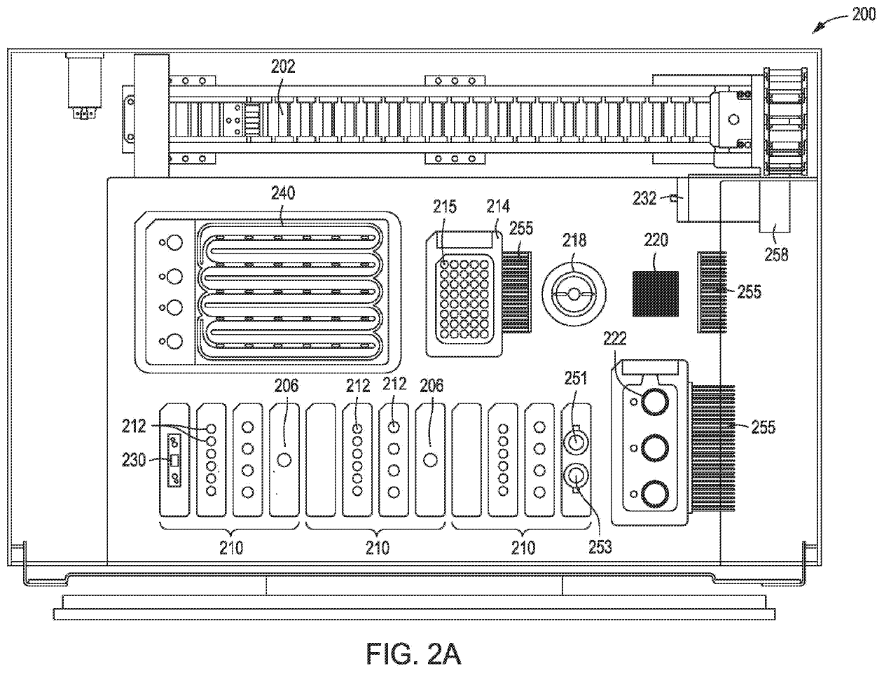

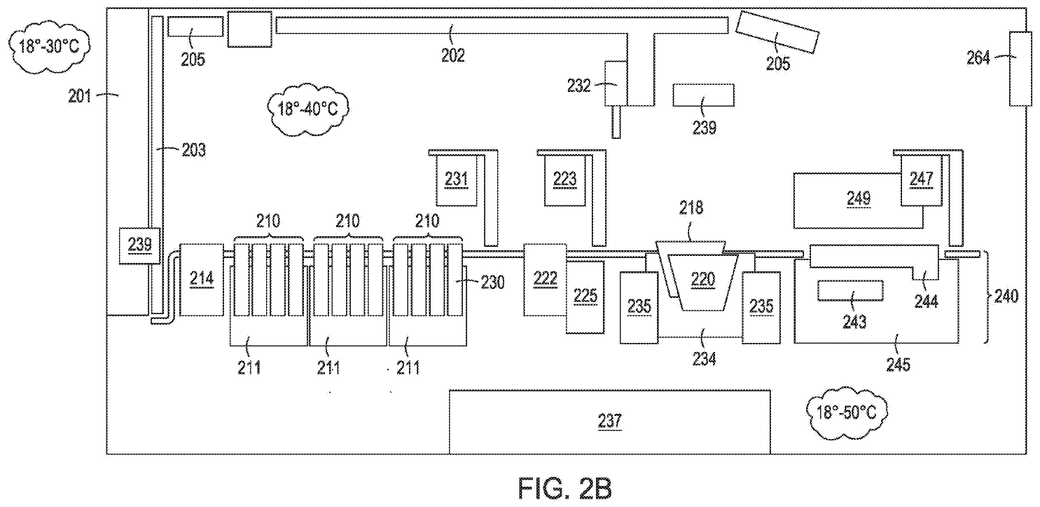

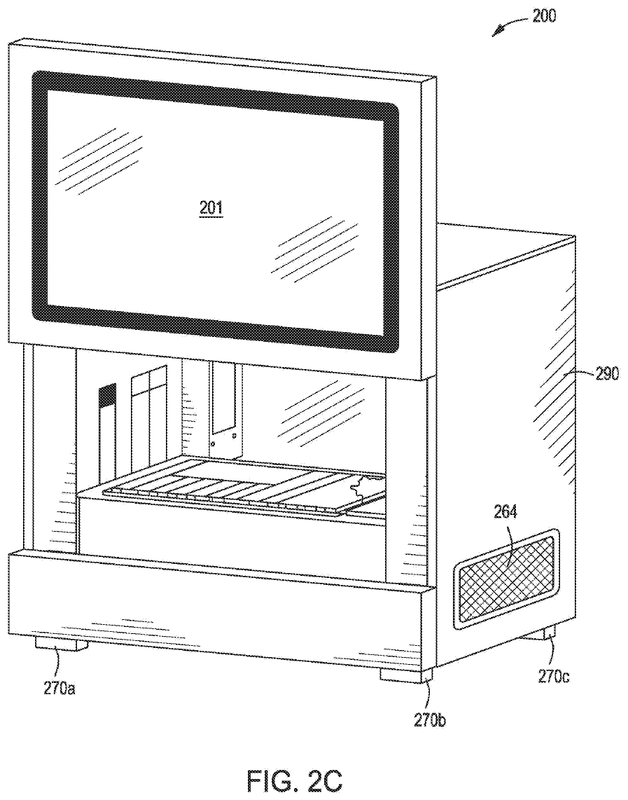

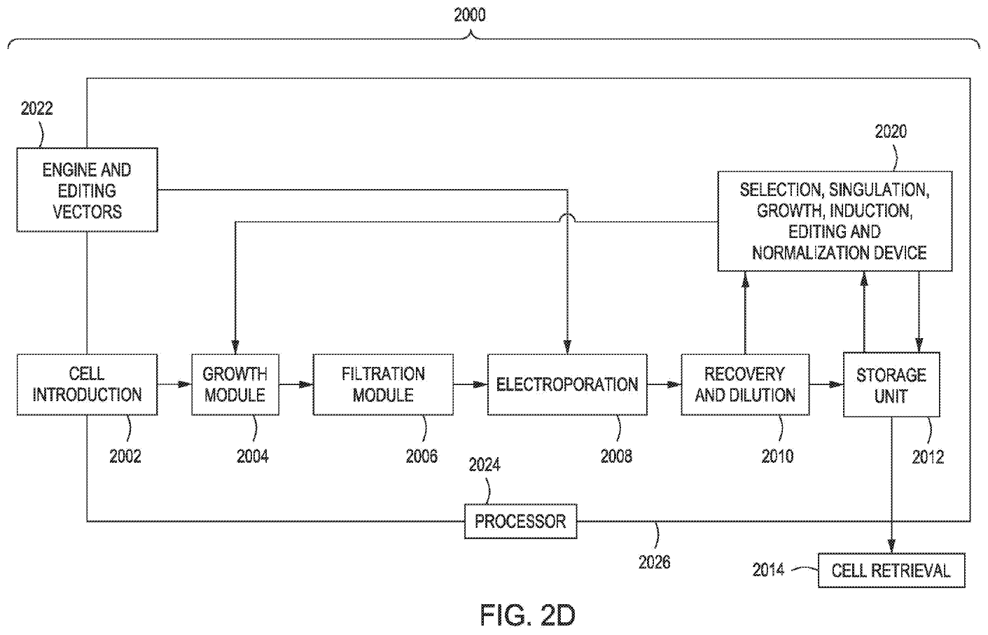

FIG. 2A depicts an example of an automated multi-module cell processing instrument 200 to, e.g., perform targeted gene editing via a fusion enzyme construct and an editing cassette comprising a binding moiety in live cells. The instrument 200, for example, may be and preferably is designed as a stand-alone benchtop instrument for use within a laboratory environment. The instrument 200 may incorporate a mixture of reusable and disposable components for performing the various integrated processes in conducting automated genome cleavage and/or editing in cells without human intervention. Illustrated is a gantry 202, providing an automated mechanical motion system (actuator) (not shown) that supplies XYZ axis motion control to, e.g., an automated (e.g., robotic) liquid handling system 258 including, e.g., an air displacement pipettor 232 which allows for cell processing among multiple modules without human intervention. In some automated multi-module cell processing instruments, the air displacement pipettor 232 is moved by gantry 202 and the various modules and reagent cartridges remain stationary; however, in other aspects, the liquid handling system 258 may stay stationary while the various modules and reagent cartridges are moved. Also included in the automated multi-module cell processing instrument 200 are reagent cartridges 210 (see, U.S. Pat. Nos. 10,376,889; 10,406,525; 10,478,822; 10,576,474; 10,639,637; 10,738,271; and 10,799,868) comprising reservoirs 212 and transformation module 230 (e.g., a flow-through electroporation (FTEP) device as described in U.S. Pat. Nos. 10,435,713; 10,443,074; and 10,851,389), as well as wash reservoirs 206, cell input reservoir 251 and cell output reservoir 253. The wash reservoirs 206 may be configured to accommodate large tubes, for example, wash solutions, or solutions that are used often throughout an iterative process. Although two of the reagent cartridges 210 comprise a wash reservoir 206 in FIG. 2A, the wash reservoirs instead could be included in a wash cartridge where the reagent and wash cartridges are separate cartridges. In such a case, the reagent cartridge and wash cartridge may be identical except for the consumables (reagents or other components contained within the various inserts) inserted therein.

In some implementations, the reagent cartridges 210 are disposable kits comprising reagents and cells for use in the automated multi-module cell processing/editing instrument 200. For example, a user may open and position each of the reagent cartridges 210 comprising various desired inserts and reagents within the chassis of the automated multi-module cell editing instrument 200 prior to activating cell processing. Further, each of the reagent cartridges 210 may be inserted into receptacles in the chassis having different temperature zones appropriate for the reagents contained therein.

Also illustrated in FIG. 2A is the robotic liquid handling system 258 including the gantry 202 and air displacement pipettor 232. In some examples, the robotic handling system 258 may include an automated liquid handling system such as those manufactured by Tecan Group Ltd. of Mannedorf, Switzerland, Hamilton Company of Reno, NV, USA (see, e.g., WO2018015544A1), or Beckman Coulter, Inc. of Fort Collins, CO, USA (see, e.g., US20160018427A1). Pipette tips 215 may be provided in a pipette transfer tip supply 214 for use with the air displacement pipettor 232. The robotic liquid handling system allows for the transfer of liquids between modules without human intervention.

Inserts or components of the reagent cartridges 210, in some implementations, are marked with machine-readable indicia (not shown), such as bar codes, for recognition by the robotic handling system 258. For example, the robotic liquid handling system 258 may scan one or more inserts within each of the reagent cartridges 210 to confirm contents. In other implementations, machine-readable indicia may be marked upon each reagent cartridge 210, and a processing system (not shown, but see element 237 of FIG. 2B) of the automated multi-module cell editing instrument 200 may identify a stored materials map based upon the machine-readable indicia. In the aspect illustrated in FIG. 2A, a cell growth module comprises a cell growth vial 218 (for details, see U.S. Pat. Nos. 10,435,662; 10,433,031; 10,590,375; 10,717,959; and 10,883,095). Additionally seen is a tangential flow filtration (TFF) module 222 (for details, see U.S. Ser. Nos. 16/516,701 and 16/798,302). Also illustrated as part of the automated multi-module cell processing instrument 200 of FIG. 2A is a singulation module 240 (e.g., a solid wall isolation, incubation and normalization device (SWIIN device) is shown here and described in detail in U.S. Pat. Nos. 10,533,152; 10,633,626; 10,633,627; 10,647,958; 10,723,995; 10,801,008; 10,851,339; 10,954,485; 10,532,324; 10,625,212; 10,774,462; and 10,835,869), served by, e.g., robotic liquid handing system 258 and air displacement pipettor 232. Additionally seen is a selection module 220 which may employ magnet separation. Also note the placement of three heatsinks 255.

FIG. 2B is a simplified representation of the contents of the example of a multi-module cell processing instrument 200 depicted in FIG. 2A. Cartridge-based source materials (such as in reagent cartridges 210), for example, may be positioned in designated areas on a deck of the instrument 200 for access by an air displacement pipettor 232 on gantry 202. The deck of the multi-module cell processing instrument 200 may include a protection sink (not shown) such that contaminants spilling, dripping, or overflowing from any of the modules of the instrument 200 are contained within a lip of the protection sink. Also seen are reagent cartridges 210, which are shown disposed with thermal assemblies 211 which can create temperature zones appropriate for different reagents in different regions. Note that one of the reagent cartridges also comprises a flow-through electroporation device 230 (FTEP), served by FTEP interface (e.g., manifold arm) and actuator 231. Also seen is TFF module 222 with adjacent thermal assembly 225, where the TFF module is served by TFF interface (e.g., manifold arm) and actuator 223. Thermal assemblies 225, 235, and 245 encompass thermal electric devices such as Peltier devices, as well as heatsinks, fans and coolers. The rotating growth vial 218 is within a growth module 234, where the growth module is served by two thermal assemblies 235. A selection module is seen at 220.

Also seen is the SWIIN module 240, comprising a SWIIN cartridge 244, where the SWIIN module also comprises a thermal assembly 245, cooling grate 264, illumination 243 (in this aspect, backlighting), evaporation and condensation control 249, and where the SWIIN module is served by SWIIN interface (e.g., manifold arm) and actuator 247. Also seen in this view is touch screen display 201, display actuator 203, illumination 205 (one on the side of multi-module cell processing instrument 200), and cameras 239 (one camera on either side of multi-module cell processing instrument 200). Finally, element 237 comprises electronics, such as a processor (237), circuit control boards, high-voltage amplifiers, power supplies, and power entry; as well as pneumatics, such as pumps, valves and sensors.

FIG. 2C illustrates a front perspective view of multi-module cell processing instrument 200 for use in as a benchtop version of the automated multi-module cell editing instrument 200. For example, a chassis 290 may have a width of about 24-48 inches, a height of about 24-48 inches and a depth of about 24-48 inches. Chassis 290 may be and preferably is designed to hold all modules and disposable supplies used in automated cell processing and to perform all processes required without human intervention; that is, chassis 290 is configured to provide an integrated, stand-alone automated multi-module cell processing instrument. As illustrated in FIG. 2C, chassis 290 includes touch screen display 201, cooling grate 264, which allows for air flow via an internal fan (not shown). The touch screen display provides information to a user regarding the processing status of the automated multi-module cell editing instrument 200 and accepts inputs from the user for conducting the cell processing. In this aspect, the chassis 290 is lifted by adjustable feet 270a, 270b, 270c and 270d (feet 270a-270c are shown in this FIG. 2C). Adjustable feet 270a-270d, for example, allow for additional air flow beneath the chassis 290.

Inside the chassis 290, in some implementations, will be most or all of the components described in relation to FIGS. 2A and 2B, including the robotic liquid handling system disposed along a gantry, reagent cartridges 210 including a flow-through electroporation device, a rotating growth vial 218 in a cell growth module 234 (see FIG. 2B), a tangential flow filtration module 222, a SWIIN module 240 as well as interfaces and actuators for the various modules. In addition, chassis 290 houses control circuitry, liquid handling tubes, air pump controls, valves, sensors, thermal assemblies (e.g., heating and cooling units) and other control mechanisms. For examples of multi-module cell editing instruments, see U.S. Pat. Nos. 10,253,316; 10,329,559; 10,323,242; 10,421,959; 10,465,185; 10,519,437; 10,584,333; 10,584,334; 10,647,982; 10,689,645; 10,738,301; 10,738,663; 10,947,532; 10,894,958; 10,954,512; and 11,034,953, all of which are herein incorporated by reference in their entirety.

FIG. 2D illustrates an aspect of a multi-module cell processing instrument. This aspect depicts an example of a system that performs recursive gene editing on a cell population. The cell processing instrument 2000 may include a housing 2026, a reservoir for storing cells to be transformed or transfected 2002, and a cell growth module (comprising, e.g., a rotating growth vial) 2004. The cells to be transformed are transferred from a reservoir 2002 to the cell growth module 2004 to be cultured until the cells hit a target OD. Once the cells hit the target OD, the growth module may cool or freeze the cells for later processing or transfer the cells to a cell concentration (e.g., filtration) module 2006 where the cells are subjected to buffer exchange and rendered electrocompetent and the volume of the cells may be reduced substantially. Once the cells have been concentrated to an appropriate volume, the cells are transferred to electroporation device 2008 or other transformation module. In addition to the reservoir for storing cells 2002, the multi-module cell processing instrument includes a reservoir for storing the engine and editing vectors or engine+editing vectors or vectors and fusion enzyme constructs to be introduced into the electrocompetent cell population 2022. The vectors are transferred to the electroporation device 2008, which already contains the cell culture grown to a target OD. In the electroporation device 2008, the nucleic acids (or nucleic acids and proteins) are electroporated into the cells. Following electroporation, the cells are transferred into an optional recovery and dilution module 2010, where the cells recover briefly post-transformation.

After recovery, the cells may be transferred to a storage module 2012, where the cells can be stored at, e.g., 4° C. or −20° C. for later processing, or the cells may be diluted and transferred to a selection/singulation/growth/induction/editing/normalization (or, e.g., SWIIN) module 2020. In the SWIIN 2020, the cells are arrayed such that there is an average of one to twenty or fifty or so cells per microwell. The arrayed cells may be in selection medium to select for cells that have been transformed or transfected with the editing vector(s). Once singulated, the cells grow through 2-50 doublings and establish colonies. Once colonies are established, editing is induced by providing conditions (e.g., temperature, addition of an inducing or repressing chemical) to induce editing. Editing is then initiated and allowed to proceed, the cells are allowed to grow to terminal size (e.g., normalization of the colonies) in the microwells and then are treated to conditions that cure the editing vector from this round. Once cured, the cells can be flushed out of the microwells and pooled, then transferred to the storage (or recovery) unit 2012 or can be transferred back to the growth module 2004 for another round of editing. In between pooling and transfer to a growth module, there typically is one or more additional steps, such as cell recovery, medium exchange (rendering the cells electrocompetent), cell concentration (typically concurrently with medium exchange by, e.g., filtration).

Note that the selection/singulation/growth/induction/editing/normalization and curing modules may be the same module, where all processes are performed in, e.g., a solid wall device, or selection and/or dilution may take place in a separate vessel before the cells are transferred to the solid wall singulation/growth/induction/editing/normalization/editing module (or e.g., SWIIN) 2020. Similarly, the cells may be pooled after normalization, transferred to a separate vessel, and cured in the separate vessel. As an alternative to singulation in, e.g., a solid wall device, the transformed cells may be grown in—and editing can be induced in-bulk liquid (see, e.g., U.S. Ser. Nos. 16/540,767, filed 14 Aug. 2019 and Ser. No. 16/545,097, filed 20 Aug. 2019) or in singulated droplets (see U.S. Pat. No. 11,142,740). Once the putatively-edited cells are pooled, they may be subjected to another round of editing, beginning with growth, cell concentration and treatment to render electrocompetent, and transformation by yet another repair template in another editing cassette via the electroporation module 2008.

In electroporation device 2008, the cells selected from the first round of editing are transformed by a second set of editing vectors and the cycle is repeated until the cells have been transformed and edited by a desired number of, e.g., editing cassettes. The multi-module cell processing instrument exemplified in FIG. 2D is controlled by a processor 2024 configured to operate the instrument based on user input or is controlled by one or more scripts including at least one script associated with the reagent cartridge. The processor 2024 may control the timing, duration, and temperature of various processes, the dispensing of reagents, and other operations of the various modules of the instrument 2000. For example, a script or the processor may control the dispensing of cells, reagents, vectors, and editing cassettes; which editing cassettes are used for cell editing and in what order; the time, temperature and other conditions used in the recovery and expression module, the wavelength at which OD is read in the cell growth module, the target OD to which the cells are grown, and the target time at which the cells will reach the target OD. In addition, the processor may be programmed to notify a user (e.g., via an application) as to the progress of the cells in the automated multi-module cell processing instrument.

It should be apparent to one of ordinary skill in the art given the present disclosure that the process described may be recursive and multiplexed; that is, cells may go through the workflow described in relation to FIG. 2D, then the resulting edited culture may go through another (or several or many) rounds of additional editing (e.g., recursive editing) with different editing cassettes. For example, the cells from round 1 of editing may be diluted and an aliquot of the edited cells edited by editing cassette A may be combined with editing cassette B, an aliquot of the edited cells edited by editing cassette A may be combined with editing cassette C, an aliquot of the edited cells edited by editing cassette A may be combined with editing cassette D, and so on for a second round of editing. After round two, an aliquot of each of the double-edited cells may be subjected to a third round of editing, where, e.g., aliquots of each of the AB-, AC-, AD-edited cells are combined with additional editing cassettes, such as editing cassettes X. Y, and Z. That is, double-edited cells AB may be combined with and edited by editing cassettes X, Y, and Z to produce triple-edited edited cells ABX, ABY, and ABZ; double-edited cells AC may be combined with and edited by editing cassettes X, Y, and Z to produce triple-edited cells ACX, ACY, and ACZ; and double-edited cells AD may be combined with and edited by editing cassettes X, Y, and Z to produce triple-edited cells ADX, ADY, and ADZ, and so on. In this process, many permutations and combinations of edits can be executed, leading to very diverse cell populations and cell libraries.

In any recursive process, it is advantageous to “cure” the editing vectors comprising the editing cassette. “Curing” is a process in which one or more editing vectors used in the prior round of editing is eliminated from the transformed cells. Curing can be accomplished by, e.g., cleaving the editing vector(s) using a curing plasmid thereby rendering the editing vectors nonfunctional; diluting the editing vector(s) in the cell population via cell growth (that is, the more growth cycles the cells go through, the fewer daughter cells will retain the editing vector(s)), or by, e.g., utilizing a heat-sensitive origin of replication on the editing vector. The conditions for curing will depend on the mechanism used for curing; that is, in this example, how the curing plasmid cleaves the editing vector. For additional information on curing, see, e.g., U.S. Pat. Nos. 10,837,021 and 11,053,507; and U.S. Ser. Nos. 17,353,282, filed 21 Jun. 2021; and Ser. No. 17/300,518, filed 27 Jul. 2021.

### Example 9: Alternative Example of an Automated Cell Editing Instrument

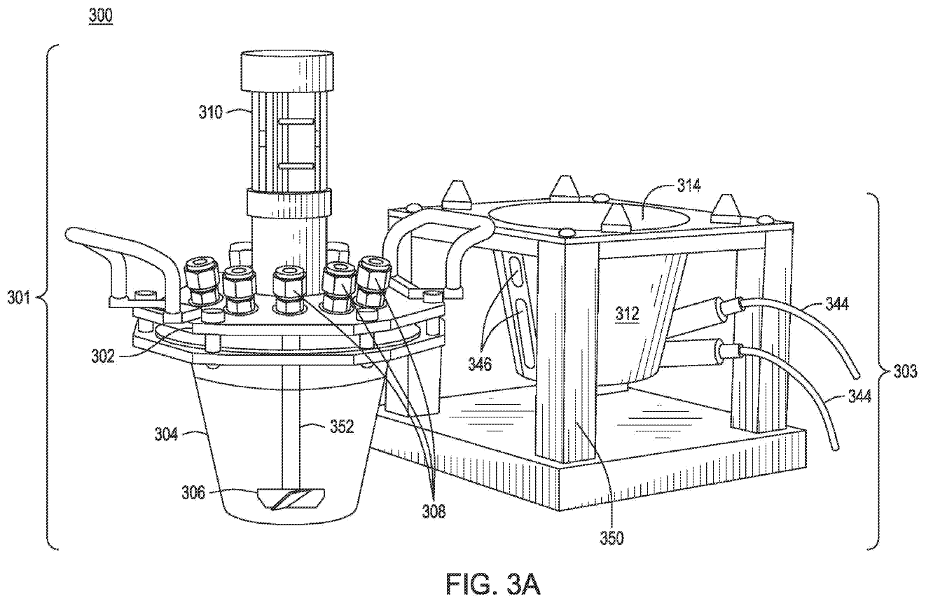

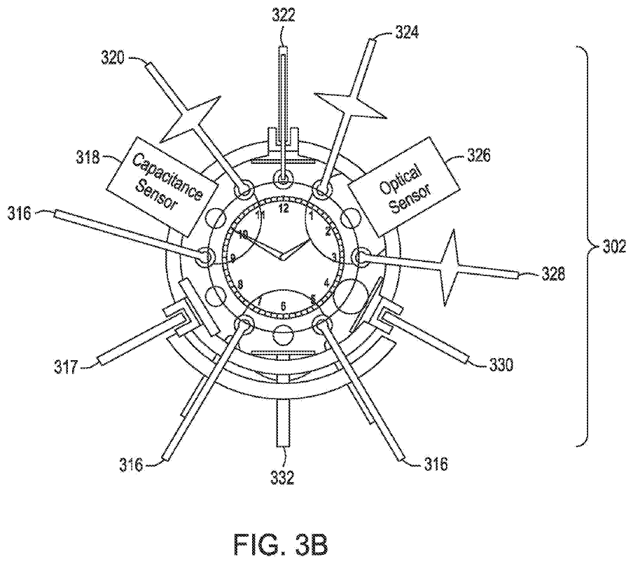

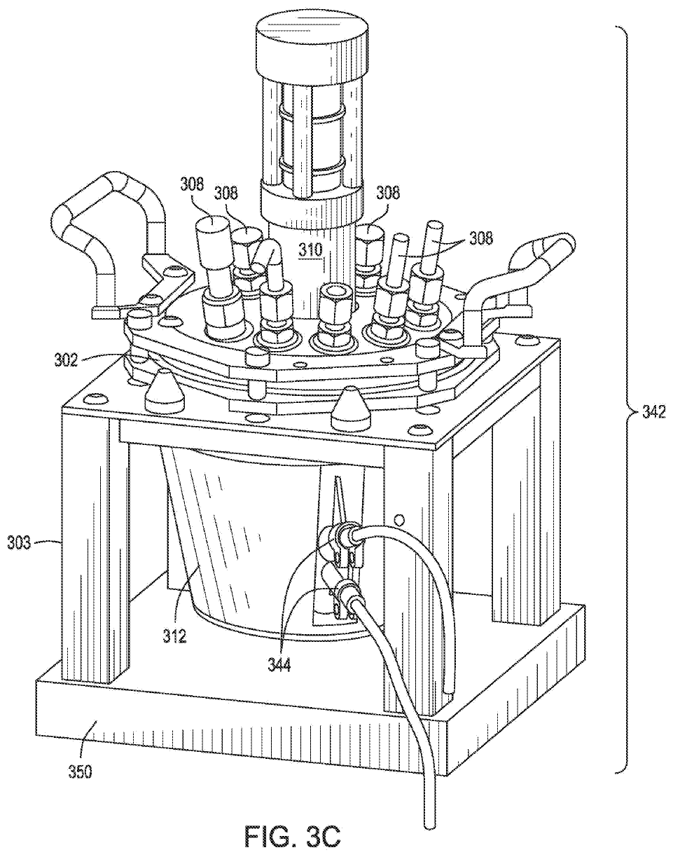

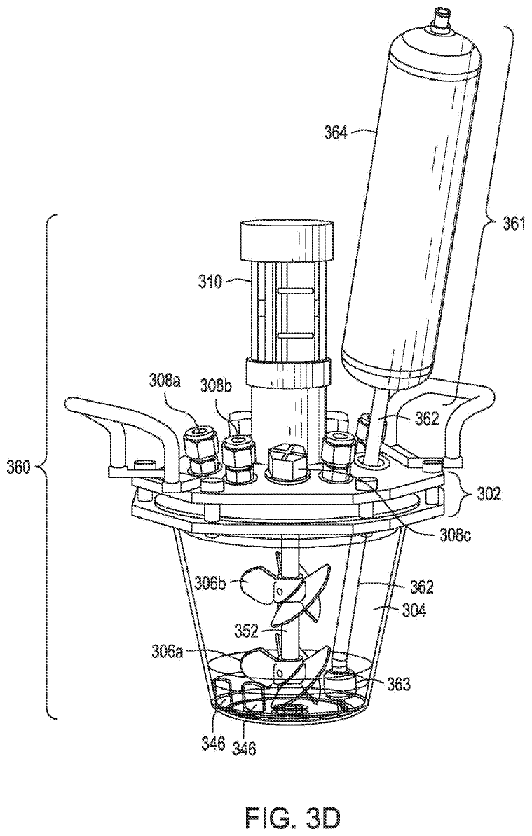

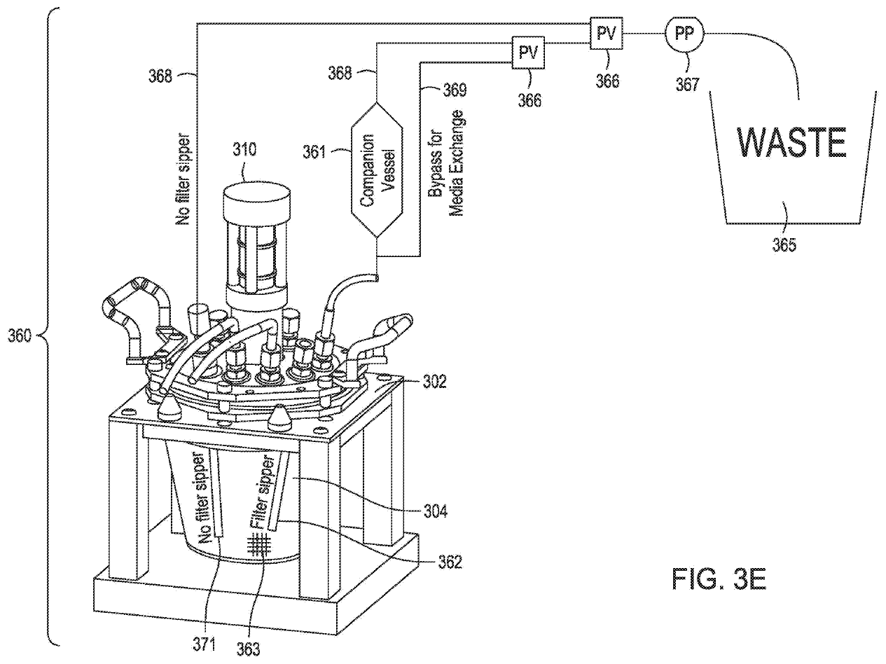

A bioreactor may be used to grow cells off-instrument or to allow for cell growth, editing and recovery on-instrument; e.g., as one module of a multi-module fully-automated closed instrument. Further, the bioreactor supports cell selection/enrichment, via expressed antibiotic markers in the growth process or via expressed antibodies coupled to magnetic beads and a magnet associated with the bioreactor. There are many bioreactors known in the art, including those described in, e.g., WO2019/046766; U.S. Pat. Nos. 10,699,519; 10,633,625; 10,577,576; 10,294,447; 10,240,117; 10,179,898; 10,370,629; and 9,175,259; and those available from Lonza Group Ltd. (Basel, Switzerland); Miltenyi Biotec (Bergisch Gladbach, Germany), Terumo BCT (Lakewood, CO, USA) and Sartorius GmbH (Gottingen, Germany). FIG. 3A shows one aspect of a bioreactor assembly 300 suitable for cell—particularly mammalian cell—growth, transfection, and fusion enzyme construct editing in the automated multi-module cell processing instruments described herein. Unlike most bioreactors that are used to support fermentation or other processes with an eye to harvesting the products produced by organisms grown in the bioreactor, the present bioreactor (and the processes performed therein) is configured to grow cells, monitor cell growth (via, e.g., optical means or capacitance), passage cells, select cells, transfect cells, and support the growth and harvesting of edited cells. Bioreactor assembly 300 comprises cell growth vessel 301 comprising a main body 304 with a lid assembly 302 comprising ports 308, including a motor integration port 310 configured to accommodate a motor to drive impeller 306 via impeller shaft 352. The tapered shape of main body 304 of the growth vessel 301 along with, in some aspects, dual impellers allows for working with a larger dynamic range of volumes, such as, e.g., up to 500 mL and as low as 100 mL for rapid sedimentation of the microcarriers. Bioreactor assembly 300 further comprises bioreactor stand assembly 303 comprising a main body 312 and growth vessel holder 314 comprising a heat jacket or other heating means (not shown) into which the main body 304 of growth vessel 301 is disposed in operation. The main body 304 of growth vessel 301 is biocompatible and preferably transparent—in some aspects, in the UV and IR range as well as the visible spectrum—so that the growing cells can be visualized by, e.g., cameras or sensors integrated into lid assembly 302 or through viewing apertures or slots 346 in the main body 312 of bioreactor stand assembly 303. Camera mounts are shown at 344. Bioreactor assembly 300 supports growth of cells from a 500,000 cell input to a 10 billion cell output, or from a 1 million cell input to a 25 billion cell output, or from a 5 million cell input to a 50 billion cell output or combinations of these ranges depending on, e.g., the size of main body 304 of growth vessel 301, the medium used to grow the cells, the type and size and number of microcarriers used for growth (if microcarriers are used), and whether the cells are adherent or non-adherent. The bioreactor that comprises assembly 300 supports growth of both adherent and non-adherent cells, wherein adherent cells are typically grown of microcarriers as described in detail in U.S. Ser. No. 17/237,747, filed 24 Apr. 2021. Alternatively, another option for growing mammalian cells in the bioreactor described herein is growing single cells in suspension using a specialized medium such as that developed by ACCELLTA™ (Haifa, Israel). Cells grown in this medium must be adapted to this process over many cell passages; however, once adapted the cells can be grown to a density of >40 million cells/mL and expanded 50-100× in approximately a week, depending on cell type.

Main body 304 of growth vessel 301 preferably is manufactured by injection molding, as is, in some aspects, impeller 306 and the impeller shaft 352. Impeller 306 also may be fabricated from stainless steel, metal, plastics or the polymers listed infra. Injection molding allows for flexibility in size and configuration and also allows for, e.g., volume markings to be added to the main body 304 of growth vessel 301. Additionally, material from which the main body 304 of growth vessel 301 is fabricated should be able to be cooled to about 4° C. or lower and heated to about 55° C. or higher to accommodate cell growth. Further, the material that is used to fabricate the vial preferably is able to withstand temperatures up to 55° C. without deformation. Suitable materials for main body 304 of growth vessel 301 include cyclic olefin copolymer (COC), glass, polyvinyl chloride, polyethylene, polyetheretherketone (PEEK), polypropylene, polycarbonate, poly(methyl) methacrylate (PMMA), polysulfone, poly(dimethylsiloxane), cyclo-olefin polymer (COP), and co-polymers of these and other polymers. Preferred materials include polypropylene, polycarbonate, or polystyrene. The material used for fabrication may depend on the cell type to be grown, transfected and edited, and be conducive to growth of both adherent and non-adherent cells and workflows involving microcarrier-based transfection. The main body 304 of growth vessel 301 may be reusable or, alternatively, may be manufactured and configured for a single use. In one aspects, main body 304 of growth vessel 301 may support cell culture volumes of 25 mL to 500 mL, but may be scaled up to support cell culture volumes of up to 3 L.

The bioreactor stand assembly comprises a stand or frame 350, a main body 312 which holds the growth vessel 301 during operation. The stand/frame 350 and main body 312 are fabricated from stainless steel, other metals, or polymer/plastics. The bioreactor stand assembly main body further comprises a heat jacket (not seen in FIG. 3A) to maintain the growth vessel main body 304—and thus the cell culture—at a desired temperature. Additionally, the stand assembly can host a set of sensors and cameras (camera mounts are shown at 344) to monitor cell culture.

FIG. 3B depicts a top-down view of one aspect of vessel lid assembly 302. Growth vessel lid assembly 302 is configured to be air-tight, providing a sealed, sterile environment for cell growth, transfection and editing as well as to provide biosafety in a closed system. Vessel lid assembly 302 and the main body 304 of growth vessel 301 (not shown here but on FIG. 3A) can be reversibly sealed via fasteners such as screws, or permanently sealed using biocompatible glues or ultrasonic welding. Vessel lid assembly 302 in some aspects is fabricated from stainless steel such as S316L stainless steel but may also be fabricated from metals, other polymers (such as those listed supra) or plastics. As seen in this FIG. 3B—as well as in FIG. 3A—vessel lid assembly 302 comprises a number of different ports to accommodate liquid addition and removal; gas addition and removal; for insertion of sensors to monitor culture parameters (described in more detail infra); to accommodate one or more cameras or other optical sensors; to provide access to the main body 304 of growth vessel 301 by, e.g., a liquid handling device; and to accommodate a motor for motor integration to drive one or more impellers 306. An example of ports depicted in FIG. 3B include three liquid-in ports 316 (at 5 o'clock, 7 o'clock and 9 o'clock), one liquid-out port 322 (at 12 o'clock), a capacitance sensor 318 (at 10 o'clock), one “gas in” port 324 (at 1 o'clock), one “gas out” port 320 (at 11 o'clock), an optical sensor 326 (at 2 o'clock), a rupture disc 328 at 3 o'clock, two self-sealing ports 317, 330 (at 8 o'clock and 4 o'clock) to provide access to the main body 304 of growth vessel 301; and (a temperature probe 332 (at 6 o'clock).

The ports shown in vessel lid assembly 302 in this FIG. 3B are examples only and it should be apparent to one of ordinary skill in the art given the present disclosure that, e.g., a single liquid-in port 316 could be used to accommodate addition of all liquids to the cell culture rather than having a liquid-in port for each different liquid added to the cell culture. Similarly, there may be more than one gas-in port 324, such as one for each gas, e.g., O₂, CO₂ that may be added. In addition, although a temperature probe 332 is shown, a temperature probe alternatively may be located on the outside of vessel holder 314 of bioreactor stand assembly separate from or integrated into heater jacket (314, 302 not seen in this FIG. 3B). One or more self-sealing ports 317, 330, if present, allow access to the main body 304 of growth vessel 301 for, e.g., a pipette, syringe, or other liquid delivery system via a gantry (not shown). As shown in FIG. 3A, additionally there may be a motor integration port 310 to drive the impeller(s), although other configurations of growth vessel 301 may alternatively integrate the motor drive at the bottom of the main body 304 of growth vessel 301. Growth vessel lid assembly 302 may also comprise a camera port for viewing and monitoring the cells.

Additional sensors include those that detect dissolved O₂ concentration, dissolved CO₂ concentration, culture pH, lactate concentration, glucose concentration, biomass, and optical density. The sensors may use optical (e.g., fluorescence detection), electrochemical, or capacitance sensing and either be reusable or configured and fabricated for single-use. Sensors appropriate for use in the bioreactor are available from Omega Engineering (Norwalk, CT, USA); PreSens Precision Sensing (Regensburg, Germany); C-CIT Sensors AG (Waedenswil, Switzerland), and ABER Instruments Ltd. (Alexandria, VA, USA). In one aspect, optical density is measured using a reflective optical density sensor to facilitate sterilization, improve dynamic range and simplify mechanical assembly.

The rupture disc, if present, provides safety in a pressurized environment, and is programmed to rupture if a threshold pressure is exceeded in growth vessel. If the cell culture in the growth vessel is a culture of adherent cells, microcarriers may be used as described in U.S. Ser. No. 17/237,747, filed 24 Apr. 2021 and as shown in FIGS. 4A-4D and described in the related text. In such an instance, the liquid-out port may comprise a filter such as a stainless steel or plastic (e.g., polyvinylidene difluoride (PVDF), nylon, polypropylene, polybutylene, acetal, polyethylene, or polyamide) filter or frit to prevent microcarriers from being drawn out of the culture during, e.g., medium exchange, but to allow dead cells to be withdrawn from the vessel. Additionally, a liquid port may comprise a filter sipper to allow cells that have been dissociated from microcarriers to be drawn into the cell corral while leaving spent microcarriers in main body of the growth vessel. The microcarriers used for initial cell growth can be nanoporous (where pore sizes are typically <20 nm in size), microporous (with pores between >20 nm to <1 μm in size), or macroporous (with pores between >1 μm in size, e.g. 20 μm) and the microcarriers are typically 50-200 μm in diameter; thus the pore size of the filter or frit in the liquid-out port will differ depending on microcarrier size.

The microcarriers used for cell growth depend on cell type and desired cell numbers, and typically include a coating of a natural or synthetic extracellular matrix or cell adhesion promoters (e.g., antibodies to cell surface proteins or poly-L-lysine) to promote cell growth and adherence. Microcarriers for cell culture are widely commercially available from, e.g., Millipore Sigma, (St. Louis, MO, USA); ThermoFisher Scientific (Waltham, MA, USA); Pall Corp. (Port Washington, NY, USA); GE Life Sciences (Marlborough, MA, USA); and Corning Life Sciences (Tewkesbury, MA, USA). As for the extracellular matrix, natural matrices include collagen, fibrin and vitronectin (available, e.g., from ESBio, Alameda, CA, USA), and synthetic matrices include MATRIGEL® (Corning Life Sciences, Tewkesbury, MA, USA), GELTREX™ (ThermoFisher Scientific, Waltham, MA, USA), CULTREX® (Trevigen, Gaithersburg, MD, USA), biomimetic hydrogels available from Cellendes (Tubingen, Germany); and tissue-specific extracellular matrices available from Xylyx (Brooklyn, NY, USA); further, denovoMatrix (Dresden, Germany) offers screenMATRIX™, a tool that facilitates rapid testing of a large variety of cell microenvironments (e.g., extracellular matrices) for optimizing growth of the cells of interest.

FIG. 3C is a side perspective view of the assembled bioreactor 342 without sensors mounted in ports 308. Seen are vessel lid assembly 302, bioreactor stand assembly 303, bioreactor stand main body 312 into which the main body of growth vessel 301 (not seen in this FIG. 3C) is inserted. Also present are two camera mounts 344, motor integration port 310 and base 350.

FIG. 3D shows the aspect of a bioreactor/cell corral assembly 360, comprising the bioreactor assembly 300 (not shown in this FIG. 3D) for cell growth, transfection, and editing described in FIG. 3A and further comprising a cell corral 361. Bioreactor assembly comprises a growth vessel comprising tapered a main body 304 with a lid assembly 302 comprising ports 308a, 308b, and 308c, including a motor integration port 310 driving impellers 306a, 306b via impeller shaft 352, as well as two viewing ports 346. Cell corral 361 comprises a main body 364, end caps, where the end cap proximal the bioreactor assembly 300 is coupled to a filter sipper 362 comprising a filter portion 363 disposed within the main body 304 of the bioreactor assembly 300 (not shown in this FIG. 3D). The filter sipper is disposed within the main body 304 of the bioreactor assembly 300 but does not reach to the bottom surface of the bioreactor assembly 300 to leave a “dead volume” for spent microcarriers to settle while cells are removed from the growth vessel 301 into the cell corral 361. The cell corral may or may not comprise a temperature or CO₂ probe, and may or not be enclosed within an insulated jacket.

The cell corral 361, like the main body 304 of growth vessel is fabricated from any biocompatible material such as polycarbonate, cyclic olefin copolymer (COC), glass, polyvinyl chloride, polyethylene, polyetheretherketone (PEEK), polypropylene, poly(methyl methacrylate (PMMA)), polysulfone, poly(dimethylsiloxane), cyclo-olefin polymer (COP), and co-polymers of these and other polymers. Likewise, the end caps are fabricated from a biocompatible material such as polycarbonate, cyclic olefin copolymer (COC), glass, polyvinyl chloride, polyethylene, polyetheretherketone (PEEK), polypropylene, poly(methyl methacrylate (PMMA)), polysulfone, poly(dimethylsiloxane), cyclo-olefin polymer (COP), and co-polymers of these and other polymers. The cell corral may be coupled to or integrated with one or more devices, such as a flow cell where an aliquot of the cell culture can be counted. Additionally, the cell corral may comprise additional liquid ports for adding medium, other reagents, and/or fresh microcarriers to the cells in the cell corral. The volume of the main body 364 of the cell corral 361 may be from 25 mL to 3000 mL, or from 250 mL to 1000 mL, or from 450 mL to 500 mL.

In operation, the bioreactor/cell corral assembly 360 comprising the bioreactor assembly 300 (not shown in this FIG. 3D) and cell corral 361 grows, passages, transfects, and supports editing and further growth of mammalian cells (note, the bioreactor stand assembly is not shown in this FIG. 3D). Cells are transferred to the growth vessel comprising medium and microcarriers. The cells are allowed to adhere to the microcarriers. Approximately 2,000,000 microcarriers (e.g., laminin-521 coated polystyrene with enhanced attachment surface treatment) are used for the initial culture of approximately 20 million cells to where there are approximately 50 cells per microcarrier. The cells are grown until there are approximately 500 cells per microcarrier. For medium exchange, the microcarriers comprising the cells are allowed to settle and spent medium is aspirated via a sipper filter, wherein the filter has a mesh small enough to exclude the microcarriers. The mesh size of the filter will depend on the size of the microcarriers and cells present but typically is from 50 μm to 500 μm, or from 70 μm to 200 μm, or from 80 μm to 110 μm. For passaging the cells, the microcarriers are allowed to settle and spent medium is removed from the growth vessel, and phosphobuffered saline or another wash agent is added to the growth vessel to wash the cells on the microcarriers. Optionally, the microcarriers are allowed to settle once again, and some of the wash agent is removed. At this point, the cells are dissociated from the microcarriers. Dissociation may be accomplished by, e.g., bubbling gas or air through the wash agent in the growth vessel, by increasing the impeller speed and/or direction, by enzymatic action (via, e.g., trypsin), or by a combination of these methods. In one aspect, a chemical agent such as the RelesRT™ reagent (STEMCELL Technologies Canada INC., Vancouver, BC, Canada) is added to the microcarriers in the remaining wash agent for a period of time required to dissociate most of the cells from the microcarriers, such as from 1 minute to 60 minutes, or from 3 minutes to 25 minutes, or from 5 minutes to 10 minutes. Once enough time has passed to dissociate the cells, cell growth medium is added to the growth vessel to stop the enzymatic reaction.

Once again, the now-spent microcarriers are allowed to settle to the bottom of the growth vessel and the cells are aspirated through a filter sipper into the cell corral 361. The growth vessel is configured to allow for a “dead volume” of 2 mL to 200 mL, or 6 mL to 50 mL, or 8 mL to 12 mL below which the filter sipper does not aspirate medium to ensure the settled spent microcarriers are not transported to the filter sipper during fluid exchanges. Once the cells are aspirated from the bioreactor vessel leaving the “dead volume” of medium and spent microcarriers, the spent microcarriers are aspirated through a non-filter sipper into waste. The spent microcarriers (and the bioreactor vessel) are diluted in phosphobuffered saline or other buffer one or more times, wherein the wash agent and spent microcarriers continue to be aspirated via the non-filter sipper leaving a clean bioreactor vessel. After washing, fresh microcarriers or RBMCs and fresh medium are dispensed into the bioreactor vessel and the cells in the cell corral are dispensed back into the bioreactor vessel for another round of passaging or for transfection and editing, respectively.

FIG. 3E depicts a bioreactor and bioreactor/cell corral assembly 360 comprising a growth vessel, with a main body 304, lid assembly 302 comprising a motor integration port 310, a filter sipper 362 comprising a filter 363 and a no-filter sipper 371. Also seen is a cell corral 361, fluid lines 368 from the cell corral through pinch valve 366, and a line 369 for medium exchange also connected to a pinch valve 366. The no-filter sipper 368 also runs through a pinch valve 366 to waste 365. Also seen is a peristaltic pump 367. For more detailed information on bioreactors and cell corrals, see U.S. Ser. No. 17/239,540, filed 24 Apr. 2021.

### Example 10: Delivery of Reagent Bundles to Mammalian Cells in a Bioreactor

FIG. 4A depicts an example workflow employing microcarrier-partitioned delivery for editing mammalian cells grown in suspension where the cells are co-localized on reagent bundle microcarriers (“RBMCs”) comprising the fusion enzyme editing components to be transfected into the cell. In a first step, the cells to be edited are grown for several passages, e.g., off instrument, to assure cell health. The cells may be grown in 2D culture, in 3D culture (if the cells are viable when grown in or adapted to 3D culture) or on microcarriers. This initial cell growth typically takes place off the automated instrument. If necessary, the cells are dissociated and added to medium in the bioreactor comprising cell growth medium such as MEM, DMEM, RPMI, or, for stem cells, mTeSR™ Plus serum-free, feeder-free cell culture medium (STEMCELL Technologies Canada INC., Vancouver, BC, Canada) and cell growth microcarriers. If the cells are grown initially on microcarriers, the microcarriers are transferred to the bioreactor comprising cell growth medium such as mTeSR™ Plus serum-free, feeder-free cell culture medium (STEMCELL Technologies Canada INC., Vancouver, BC, Canada) and additional microcarriers. Approximately 1 e7 or 1 e8 cells are transferred to the cell growth module on the automated instrument for growth.

In parallel with the off-instrument cell growth, reagent bundle microcarriers (RBMCs) are manufactured, also off-instrument. The present description provides depictions of two examples of methods where several steps involve manufacturing RBMCs (see FIGS. 4C and 4D) that may be used to edit the cells in the modules and automated instruments described herein.

The cells are grown in 3D culture on microcarriers in the bioreactor for, e.g., three to four days or until a desired number of cells, e.g., 1 e8, cells are present. Note that all processes in this FIG. 4A may take place in the bioreactor and cell corral. During this growth cycle, the cells are monitored for cell number, pH, and optionally other parameters. As described above, cell growth monitoring can be performed by imaging, for example, by allowing the microcarriers to settle and imaging the bottom of the bioreactor. Alternatively, an aliquot of the culture may be removed and run through a separate flow cell, e.g., in a separate module, for imaging. For example, the cell corral, in addition to being integrated with the bioreactor vessel, may be integrated with a flow cell or other device for cell counting where an aliquot of the cell culture in the cell corral may be removed and counted in the flow cell.

In another alternative, the cells may express a fluorescent protein and fluorescence in the cell culture is measured or fluorescent dye may be used to stain cells, particularly live cells. This microcarrier-based workflow can be performed in the bioreactor and cell corral with most if not all steps performed in the same device; thus, several bioreactors and cell corrals may be deployed in parallel for two to many samples simultaneously. In yet another alternative, permittivity or capacitance is used to monitor cell coverage on the microcarriers. In yet another aspect, an aliquot of cells may be removed from the bioreactor or cell corral and transported out of the instrument and manually counted on a commercial cell counter (e.g., Thermofisher Countess, Waltham, MA, USA).

The microcarriers used for initial cell growth can be nonporous (where pore sizes are typically <20 nm in size), microporous (with pores between >20 nm to <1 μm in size), or macroporous (with pores between >1 μm in size, e.g. 20 μm). In microcarrier culture, cells grow as monolayers on the surface of nonporous or microporous microcarriers, which are typically spherical in morphology; alternatively, the cells grow on the surface and as multilayers in the pores of macroporous microcarriers. The microcarriers preferably have a density slightly greater than that of the culture medium to facilitate easy separation of cells and medium for, e.g., medium exchange and imaging and passaging; yet the density of the microcarriers is also sufficiently low to allow complete suspension of the microcarriers at a minimum stirring or bubbling rate. Maintaining a low stirring or bubbling rate is preferred so as to avoid hydrodynamic damage to the cells.

The microcarriers used for cell growth depend on cell type and desired cell numbers, and typically include a coating of a natural or synthetic extracellular matrix or cell adhesion promoters (e.g., antibodies to cell surface proteins or poly-L-lysine) to promote cell growth and adherence. Microcarriers for cell culture are widely commercially available from, e.g., Millipore Sigma, (St. Louis, MO, USA); Thermo Fisher (Waltham, MA, USA); Pall Corp. (Port Washington, NY, USA); GE Life Sciences (Marlborough, MA, USA); and Corning Life Sciences (Tewkesbury, MA, USA). As for the extracellular matrix, natural matrices include collagen, fibrin and vitronectin (available, e.g., from ESBio, Alameda, CA, USA), and synthetic matrices include Matrigel® (Corning Life Sciences, Tewkesbury, MA, USA), Geltrex™ (Thermo Fisher Scientific, Waltham, MA, USA), Cultrex® (Trevigen, Gaithersburg, MD, USA), biomimetic hydrogels available from Cellendes (Tubingen, Germany); and tissue-specific extracellular matrices available from Xylyx (Brooklyn, NY, USA); further, denovoMatrix (Dresden, Germany) offers screenMATRIX™, a tool that facilitates rapid testing of a large variety of cell microenvironments (e.g., extracellular matrices) for optimizing growth of the cells of interest.

Following cell growth, passaging is performed by, e.g., stopping the impeller rotation or bubbling action in the bioreactor and allowing the microcarriers to settle. In one method, the cells are removed from the microcarriers using enzymes such as collagenase, trypsin or pronase, or by non-enzymatic methods including EDTA or other chelating chemicals, and once removed from the carriers, medium is added to dilute the enzyme to inhibit enzymatic action. The dissociation procedures relating to the cell corral are described in detail infra. Once medium is added, then the cells are separated from the microcarriers by allowing the microcarriers to settle and aspirating the cells via a filtered sipper into the cell corral. The cells then may be optionally dissociated from one another via a filter, sieve or by bubbling or other agitation in the cell corral. Next, microcarriers comprising the manufactured reagent bundles with the fusion enzyme editing components (reagent bundle microcarrier microcarriers or RBMCs) and the dissociated cells are combined in an appropriate medium in the growth vessel. Alternatively, instead of removing cells from the cell growth microcarriers and re-seeding on RBMCs, the cells may be transferred from the cell growth microcarriers to RBMCs via microcarrier bridge passaging either in the growth vessel in a reduced volume or in the cell corral. Bridge passaging involves allowing a new microcarrier (e.g. an RBMC) to come into physical contact with a cell-laden microcarrier, such that cells on the latter microcarrier can migrate to the RBMC.

RBMCs are not prepared on-instrument but are pre-manufactured. The microcarriers used for reagent bundles may be microporous microcarriers, which, due to the plethora of micropores, can carry a larger reagent payload per carrier diameter than nonporous or macroporous microcarriers. Preferred microcarriers are microporous, to provide increased surface area for reagent delivery, and functionalized on the surface so as to be able to bind reagents. Preferred microcarriers for RBMCs include Pierce™ Streptavidin UltraLink™ Resin, a cross-linked polyacrylamide carrier functionalized with streptavidin comprising a pore size of 50 nm to 100 nm; Pierce™ NeutrAvidinT™ Plus UltraLink™ Resin, cross-linked polyacrylamide carrier functionalized with avidin comprising a pore size of 50 nm to 100 nm; and UltraLink™ Hydrazide Resin, a cross-linked polyacrylamide carrier functionalized with hydrazine comprising a pore size of 50 nm to 100 nm, all available from Thermo Fisher (Waltham, MA, USA); cross-linked agarose resins with alkyne, azide, photo-cleavable azide and disulfide surface functional groups available from Click Chemistry Tools (Scottsdale, AZ, USA); Sepharose™ Resin, cross-linked agarose with amine, carboxyl, carbodiimide, N-hydroxysuccinimide (NHS), and epoxy surface functional groups available from GE Health (Chicago, IL, USA). The microcarriers are loaded with amplified ECBMs or amplified editing plasmids, engine plasmids, fusion enzyme construct, fusion enzyme construct mRNAs or ribonucleoproteins (RNPs) depending on, e.g., the functionalized group, via, e.g., via chemical or photo linkage or depending on a surface coating on the microcarrier, if present. RBMCs are prepared by 1) partitioning and amplifying a single copy of an editing cassette to produce clonal copies in an RBMC, or by 2) pooling and amplifying editing cassettes, followed by dividing the editing cassettes into sub-pools and “pulling down” the amplified editing cassettes with microcarriers comprising nucleic acids specific to and complementary to unique sequences on the editing cassettes. The step of sub-pooling acts to “de-multiplex” the editing cassette pool, thereby increasing the efficiency and specificity of the “pull down” process. De-multiplexing thus allows for amplification and error correction of the editing cassettes to be performed in bulk followed by efficient loading of clonal copies of the editing cassettes onto a microcarrier.

FIG. 4B depicts an additional example for growing, passaging, transfecting and editing iPSCs (induced pluripotent stem cells), where there is sequential delivery of clonal high copy number (HCN) RBMCs—e.g., lipid nanoparticle-coated microcarriers, where each microcarrier is coated with many copies of ECBMs or editing vectors carrying a single clonal editing cassette-followed by bulk fusion enzyme construct delivery. Note that the bioreactors and cell corrals described supra may be used for all processes. First cells are seeded on the RBMCs to deliver clonal copies of editing cassettes to the cells. Again, the RBMCs comprising the ECBMs are typically fabricated or manufactured off-instrument. The cells are allowed to grow and after 24 hours to 48 hours, medium is exchanged for medium containing antibiotics to select for cells that have been transfected. The cells are passaged, re-seeded and grown again, and then passaged and re-seeded, this time onto microcarriers comprising lipofectamine with the fusion enzyme construct provided as a coding sequence under the control of a promoter, or as a protein on the surface of a microcarrier. As an alternative, the fusion enzyme construct may be provided in bulk in solution. The fusion enzyme construct is taken up by the cells on the microcarriers, and the cells are incubated and allowed to grow. Medium is exchanged as needed and the cells are detached from the microcarriers for subsequent growth and analysis. An alternative example for the method shown in FIG. 4B comprises the steps of growing, passaging, transfecting and editing iPSCs. In this aspect, there is simultaneous delivery of ECBMs RBMCs (e.g., reagent bundle lipid nanoparticle-coated microcarriers) where each microcarrier is coated with many copies of the ECBMs or editing vectors carrying a single clonal editing cassette and fusion enzyme construct (e.g., as a coding sequence under the control of a promoter therefor, as a ribonucleoprotein complex, or as a protein). Again, the RBMCs are typically fabricated or manufactured off-instrument. Note that the integrated instrument described infra may be used for all processes. As with the workflow shown in FIG. 4B, first cells are seeded on microcarriers to grow. The cells are then passaged, detached, re-seeded, grown and detached again to increase cell number, with medium exchanged every 24 hours to 48 hours or 24 hours to 72 hours as needed. Following detachment, the cells are seeded on RBMCs comprising the ECBMs for clonal delivery of the ECBMs and enzyme in a co-transfection reaction. Following transfection, the cells grown for 24 hours to 48 hours after which medium is exchanged for medium containing antibiotics for selection. The cells are selected and passaged, re-seeded and grown again. Medium is exchanged as needed and the cells are detached from the microcarriers for subsequent growth and analysis.

FIGS. 4C and 4D depict alternative methods for populating microcarriers with a lipofectamine/editing cassette payload and cells. In the method 400a shown in FIG. 4C at top left, lipofectamine 402 and ECBM payloads 404 are combined and editing LNPs (lipofectamine nucleic acid payloads) 406 are formed in solution. In parallel, microcarriers 408 (“MCs”) are combined with a coating such as laminin 521 410 to foster adsorption and cell attachment. The laminin 521-coated microcarriers are then combined with the editing LNPs 406 to form partially-loaded microcarriers 412. The processes of forming ECBM (editing) RBMCs (e.g., the partially-loaded microcarriers 412 comprising the editing LNPs 406) to this point are typically performed off-instrument. In parallel and typically off-instrument, fusion enzyme construct LNPs 420 are formed by combining lipofectamine 402 and fusion enzyme construct mRNA 418. The fusion enzyme construct LNPs 420 are combined with the partially-loaded microcarriers 412 and adsorb onto the partially-loaded microcarriers 412 to form fully-loaded RBMCs 422 comprising both the editing (ECBM) LNPs 406 and the fusion enzyme construct LNPs 420. At this point, the mammalian cells 414 have been grown and passaged in the bioreactor and cell corral several to many times. The cells 414 populate the fully-loaded RBMCs 422, where the cells 414 then take up (e.g., are transfected by) the editing LNPs 406 and the fusion enzyme construct LNPs 420, a process that may take several hours up to several days. At the end of the transfection process, transfected mammalian cells reside on the surface of the fully-loaded microcarriers 422.

As an alternative to the method 400a shown in FIG. 4C, FIG. 4D depicts method 400b which features simultaneous adsorption of the editing (ECBM) LNPs and the fusion enzyme construct LNPs. Again, lipofectamine 402 and editing vector payloads 404 are combined where editing LNPs (lipofectamine nucleic acid payloads) 406 are formed in solution. In parallel, fusion enzyme construct LNPs 420 are formed by combining lipofectamine 402 and fusion enzyme construct mRNA 418. Also in parallel, microcarriers 408 are combined with a coating such as laminin 521 410 to foster adsorption and cell attachment. The laminin 521-coated microcarriers are simultaneously combined with both the editing LNPs 406 and the nickase LNPs 420 to form fully-loaded microcarriers 424 where both the editing LNPs 406 and the nickase LNPs 420 co-adsorb onto the surface of the laminin-coated microcarriers. The processes of forming RBMCs (i.e., the fully-loaded microcarriers 424 comprising both the editing LNPs 406 and the fusion enzyme construct LNPs 420) to this point are typically performed off-instrument.

At this point, the fully-loaded microcarriers 424 comprising the editing LNPs 406 and the fusion enzyme construct LNPs 420 are added to medium in the bioreactor comprising the mammalian cells 414 to be transfected, optionally with additional lipofect reagent 402. The mammalian cells 414 have been grown and passaged in the bioreactor and cell corral one to many times. The cells 414 populate the fully-loaded RBMCs 424, where the cells 414 then take up (e.g., are transfected by) the editing (ECBM) LNPs 406 and the fusion enzyme construct LNPs 420, a process that may take several hours up to several days. At the end of the transfection process, transfected mammalian cells reside on the surface of the fully-loaded microcarriers 424. In these example methods, fusion enzyme construct mRNAs are used to form the fusion enzyme construct LNPs; however, the fusion enzyme constructs may be loaded on to form LNPs, or editing cassettes and fusion enzyme constructs may be loaded in the form of ribonucleoproteins (RNPs) on the LNPs. For additional details on microcarriers and RBMCs, please see U.S. Ser. No. 17/239,540, filed 24 Apr. 2021. In some aspects, the compositions, methods, and modified cells of the current disclosure applies to the use of gRNA. In some aspects, the compositions, methods, and modified cells of the current disclosure applies to the use of any type of gRNA. In some aspects, the compositions, methods, and modified cells of the current disclosure applies to the use of one or more types of gRNAs.

In some aspects, the compositions, methods, and modified cells of the current disclosure applies to gene editing via endogenous repair mechanisms, e.g., Homology-Directed Repair (HDR), recombination pathways, or other DNA repair pathways. In some aspects, the compositions, methods, and modified cells of the current disclosure applies to HDR-based gene editing. In some aspects, the compositions, methods, and modified cells of the current disclosure applies to any method to introduce a genetic mutation into a genome (e.g., knock-in). In some aspects, the compositions, methods, and modified cells of the current disclosure applies to the use of gRNAs and HDR-based gene editing.

While this invention is satisfied by aspects in many different forms, as described in detail in connection with preferred aspects of the invention, it is understood that the present disclosure is to be considered as an example of the principles of the invention and is not intended to limit the invention to the specific aspects illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6. A variety of further modifications and improvements in and to the compositions, methods, and modified cells of the present disclosure will be apparent to those skilled in the art. The following non-limiting, embodiments are specifically envisioned:
1. A system comprising: (i) a fusion polypeptide comprising first and second orthogonal nucleases and further comprising or coupled to a recruiting moiety, (ii) a repair template comprising or coupled with a binding moiety, and optionally (iii) one or more guide RNAs; wherein the recruiting moiety recognizes the binding moiety and forms a binding pair.
2. The system of embodiment 1, wherein the system performs genome repair via homology directed repair (HDR).
3. The system of embodiment 1, wherein the system performs genome repair via non-homologous end joining (NHEJ) repair.
4. The system of embodiment 1, wherein one or both of the first and second orthogonal nucleases is an RNA-guided nuclease.
5. The system of embodiment 1, wherein one or both of the first and second orthogonal nucleases is a CRISPR nuclease.
6. The system of embodiment 1, wherein the formation of the binding pair is via non-covalent interactions.
7. The system of embodiment 1, wherein (i), (ii), and (iii), either entirely or in part, are encoded by one or more nucleic acids on one or more constructs.
8. The system of embodiment 1, wherein the repair template comprises a single-stranded oligonucleotide donor DNA (ssODN) or a double-stranded donor DNA.
9. The system of embodiment 1, wherein either one of the moieties of the binding pair is a nucleic acid, a polypeptide, a chemical modification, or any combination thereof.
10. The system of embodiment 1, wherein the repair template further comprises one or more chemical or covalent modifications.
11. The system of any one of embodiments 1 to 10, wherein the recruiting moiety is streptavidin and the binding moiety is biotin or wherein the recruiting moiety is biotin and the binding moiety is streptavidin.
12. The system of any one of embodiments 1 to 10, wherein the recruiting moiety is Epstein-Barr virus (EBV)-encoded nuclear antigen-1 (EBNA1) and the binding moiety is an origin of plasmid replication (oriP) or wherein the recruiting moiety is oriP and the binding moiety is EBNA1.
13. The system of any one of embodiments 1 to 10, wherein the recruiting moiety is SV40 T-antigen and the binding moiety is SV40 origin of replication or wherein the recruiting moiety is SV40 origin of replication and the binding moiety is SV40 T-antigen.
14. The system of any one of embodiments 1 to 10, wherein the recruiting moiety is BK T-antigen and the binding moiety is BK Virus (BKV) origin of replication or wherein the recruiting moiety is BKV origin of replication and the binding moiety is BK T-antigen.
15. The system of any one of embodiments 1 to 10, wherein the recruiting moiety is latency-associated nuclear antigen (LANA1) of Karposi's Sarcoma Herpesvirus (KSHV) and the binding moiety is LANA binding site (LBS) of KSHV or wherein the recruiting moiety is LANA binding site of KSHV and the binding moiety is LANA1 of KSHV.
16. The system of any one of embodiments 1 to 10, wherein the recruiting moiety is E2 protein of human papilloma virus (HPV) and the binding moiety is minichromosome maintenance element (MME) region of HPV or wherein the recruiting moiety is MME region of HPV and the binding moiety is E2 protein of HPV.
17. The system of any one of embodiments 1 to 10, wherein the recruiting moiety is an HUH-tag and the binding moiety is an HUH recognition sequence or wherein the recruiting moiety is an HUH recognition sequence and the binding moiety is an HUH-tag.
18. The system of any one of embodiments 1 to 10, wherein the recruiting moiety is a Udg variant and the binding moiety is uracilated DNA or wherein the recruiting moiety is uracilated DNA and the binding moiety is a Udg variant.
19. The system of any one of embodiments 1 to 10, wherein the recruiting moiety is a retron and the binding moiety is retron-synthesized RNA.
20. The system of any one of embodiments 1 to 10, wherein the recruiting moiety is positioned between the first and second orthogonal nucleases.
21. The system of any one of embodiments 1 to 10, wherein the recruiting moiety is positioned N-terminal to the first and second orthogonal nucleases.
22. The system of any one of embodiments 1 to 10, wherein the recruiting moiety is positioned C-terminal to the first and second orthogonal nucleases.
23. The system of any one of embodiments 1 to 10, wherein the first and second orthogonal nucleases are Type II nucleases.
24. The system of any one of embodiments 1 to 10, wherein the first orthogonal nuclease is Streptococcus pyogenes Cas9 (SpCas9) and the second orthogonal nuclease is Staphylococcus aureus Cas9 (SaCas9).
25. The system of any one of embodiments 1 to 10, wherein the first and second orthogonal nucleases are Type V nucleases.
26. The system of any one of embodiments 1 to 10, wherein the first orthogonal nuclease is a Type II nuclease and the second orthogonal nuclease is a Type V nuclease.
27. The system of any one of embodiments 1 to 10, wherein the one or more guide RNAs comprise a G-quadruplex (GQ)-forming sequence, wherein the repair template further comprises a corresponding GQ-forming sequence, and wherein the one or more guide RNAs is non-covalently linked to the repair template by formation of a GQ.
28. The system of any one of embodiments 1 to 10, wherein the one or more guide RNAs comprise a heteroduplex barcode sequence, wherein the repair template further comprises the reverse complement of the heteroduplex barcode sequence, and wherein the one or more guide RNA is non-covalently linked to the repair template by formation of a RNA/DNA hybrid.
29. A system comprising: (i) an RNA-guided nuclease, (ii) a guide RNA comprising or coupled to a recruiting moiety, and (iii) a repair template molecule comprising or coupled with a binding moiety; wherein the recruiting moiety recognizes the binding moiety and forms a binding pair.
30. The system of embodiment 29, wherein the system performs genome repair via homology directed repair (HDR).
31. The system of embodiment 29, wherein the system performs genome repair via non-homologous end joining (NHEJ) repair.
32. The system of embodiment 29, wherein the RNA-guided nuclease is a CRISPR nuclease.
33. The system of embodiment 29, wherein the formation of the binding pair is via non-covalent interactions.
34. The system of embodiment 29, wherein either one of the moieties of the binding pair is a DNA, an RNA, a chemical modification, or any combination thereof.
35. The system of embodiment 29, wherein (i), (ii), and (iii), either entirely or in part, are encoded by one or more nucleic acids on one or more constructs.
36. The system of embodiment 29, wherein the repair template further comprises one or more chemical or covalent modifications.
37. The system of embodiment 29, wherein the repair template comprises a single-stranded oligonucleotide donor DNA (ssODN).
38. The system of any one of embodiments 29 to 37, wherein the recruiting moiety comprises a G-quadruplex (GQ)-forming sequence.
39. The system of any one of embodiments 38, wherein the binding moiety comprises a corresponding G-quadruplex (GQ)-forming sequence.
40. The system of any one of embodiments 29 to 37, wherein the recruiting moiety comprises a heteroduplex barcode sequence.
41. The system of embodiment 40, wherein the binding moiety comprises a reverse complement of the heteroduplex barcode sequence.
42. The system of embodiment 40 or 41, wherein the heteroduplex barcode sequence is about 20 nucleotides in length.
43. The system of any one of embodiments 29 to 37, wherein the recruiting moiety is at the 3′ end of the guide RNA.
44. The system of any one of embodiments 29 to 37, wherein the binding moiety is at either the 3′ end or the 5′ end of the repair template.
45. The system of any one of embodiments 29 to 37, wherein the RNA-guided nuclease is a fusion polypeptide comprising first and second orthogonal nucleases.
46. The system of any one of embodiments 29 to 37, wherein the RNA-guided nuclease is a fusion polypeptide comprising first and second orthogonal nucleases and further comprising or coupled to a second recruiting moiety, and wherein the repair template further comprises or is coupled with a second binding moiety; wherein the second recruiting moiety recognizes the second binding moiety and forms a second binding pair.
47. A method for increasing genome repair during CRISPR editing of genomes in a population of cells comprising the steps of.
   a. designing and synthesizing a library of editing cassettes wherein each of the editing cassettes encodes a recruitment construct comprising a guide RNA, a repair template, and a binding moiety of a binding pair;
   b. designing and synthesizing a fusion enzyme construct comprising first and second orthogonal nucleases and a recruiting moiety of the binding pair;
   c. forming ribonucleoprotein (RNP) complexes with the recruitment construct and the fusion enzyme construct;
   d. introducing the RNP complexes into cells to be edited; and
   e. providing conditions for editing in the cells, wherein the recruiting moiety of the fusion enzyme construct binds to the binding moiety of the recruitment construct thereby bringing the recruitment construct and the fusion enzyme construct into proximity with one another.
48. The method of embodiment 47, wherein the genome repair is made by homology directed repair (HDR).
49. The method of embodiment 47, wherein the genome repair is made by non-homologous end joining (NHEJ) repair.
50. The method of any one of embodiments 47 to 49, wherein the recruiting moiety is streptavidin and the binding moiety is biotin or wherein the recruiting moiety is biotin and the binding moiety is streptavidin.
51. The method of any one of embodiments 47 to 49, wherein the recruiting moiety is Epstein-Barr virus (EBV)-encoded nuclear antigen-1 (EBNA1) and the binding moiety is an origin of plasmid replication (oriP) or wherein the recruiting moiety is oriP and the binding moiety is EBNA1.
52. The method of any one of embodiments 47 to 49, wherein the recruiting moiety is SV40 T-antigen and the binding moiety is SV40 origin of replication or wherein the recruiting moiety is SV40 origin of replication and the binding moiety is SV40 T-antigen.
53. The method of any one of embodiments 47 to 49, wherein the recruiting moiety is BK T-antigen and the binding moiety is BK Virus (BKV) origin of replication or wherein the recruiting moiety is BKV origin of replication and the binding moiety is BK T-antigen.
54. The method of any one of embodiments 47 to 49, wherein the recruiting moiety is latency-associated nuclear antigen (LANA1) of Karposi's Sarcoma Herpesvirus (KSHV) and the binding moiety is LANA binding site (LBS) of KSHV or wherein the recruiting moiety is LANA binding site of KSHV and the binding moiety is LANA1 of KSHV.
55. The method of any one of embodiments 47 to 49, wherein the recruiting moiety is E2 protein of human papilloma virus (HPV) and the binding moiety is minichromosome maintenance element (MME) region of HPV or wherein the recruiting moiety is MME region of HPV and the binding moiety is E2 protein of HPV.
56. The method of any one of embodiments 47 to 49, wherein the recruiting moiety is an HUH-tag and the binding moiety is an HUH recognition sequence or wherein the recruiting moiety is an HUH recognition sequence and the binding moiety is an HUH-tag.
57. The method of any one of embodiments 47 to 49, wherein the recruiting moiety is a Udg variant and the binding moiety is uracilated DNA or wherein the recruiting moiety is uracilated DNA and the binding moiety is a Udg variant.
58. The method of any one of embodiments 47 to 49, wherein the recruiting moiety is a retron and the binding moiety is retron-synthesized RNA.
59. The method of any one of embodiments 47 to 49, wherein the recruiting moiety of the binding pair is positioned between the first and second orthogonal nucleases.
60. The method of any one of embodiments 47 to 49, wherein the recruiting moiety of the binding pair is positioned N-terminal to the first and second orthogonal nucleases.
61. The method of any one of embodiments 47 to 49, wherein the recruiting moiety of the binding pair is positioned C-terminal to the first and second orthogonal nucleases.
62. The method of any one of embodiments 47 to 49, wherein the first and second orthogonal nucleases are Type II nucleases.
63. The method of embodiment 62, wherein the first orthogonal nuclease is Streptococcus pyogenes Cas9 (SpCas9) and the second orthogonal nuclease is Staphylococcus aureus Cas9 (SaCas9).
64. The method of any one of embodiments 47 to 49, wherein the first and second orthogonal nucleases are Type V nucleases.
65. The method of any one of embodiments 47 to 49, wherein the first orthogonal nuclease is a Type II nuclease and the second orthogonal nuclease is a Type V nuclease.
66. The method of any one of embodiments 47 to 49, wherein the repair template comprises a single-stranded oligonucleotide donor DNA (ssODN) or a double-stranded donor DNA.
67. The method of any one of embodiments 47 to 49, further comprising enriching for edited cells.
68. The method of embodiment 67, wherein the enriching is achieved by selecting for edited cells that express a selectable marker.
69. The method of any one of embodiments 47 to 49, wherein steps (c) to (e) is repeated to achieve one or more rounds of further editing.
70. The method of any one of embodiments 47 to 49, wherein the guide RNA comprises a G-quadruplex (GQ)-forming sequence, wherein the repair template comprises a corresponding GQ-forming sequence, and wherein the guide RNA is non-covalently linked to the repair template by formation of a GQ.
71. The method of any one of embodiments 47 to 49, wherein the guide RNA comprises a heteroduplex barcode sequence, wherein the repair template comprises the reverse complement of the heteroduplex barcode sequence, and wherein the guide RNA is non-covalently linked to the repair template by formation of a RNA/DNA hybrid.
72. A method for increasing genome repair during CRISPR editing of a genome of a live cell, comprising:
   a. providing a live cell suitable for the editing, wherein the live cell comprises ribonucleoprotein (RNP) complexes formed with a recruitment construct and a fusion enzyme construct, wherein the recruitment construct comprises a binding moiety of a binding pair and a repair template, and wherein the fusion enzyme construct comprises two orthogonal nucleases and a recruiting moiety of the binding pair;
   b. providing conditions for editing the cell, wherein the recruiting moiety of the fusion enzyme construct binds to the binding moiety of the recruitment construct thereby bringing the recruitment construct and the fusion enzyme construct into proximity with one another.
73. The method of embodiment 72, wherein the recruitment construct further comprises a guide RNA.
74. The method of embodiment 72, wherein the repair template comprises a single-stranded oligonucleotide donor DNA (ssODN) or a double-stranded donor DNA.
75. The method of embodiment 73, wherein the guide RNA comprises a GQ-forming sequence, wherein the repair template comprises a corresponding GQ-forming sequence, and wherein the guide RNA is non-covalently linked to the repair template by formation of a GQ.
76. The method of embodiment 73, wherein the guide RNA comprises a heteroduplex barcode sequence, wherein the repair template comprises the reverse complement of the heteroduplex barcode sequence, and wherein the guide RNA is non-covalently linked to the repair template by formation of a RNA/DNA hybrid.
77. The method of any one of embodiments 72 to 76, further comprising selecting for edited cells that express a selectable marker.
78. The method of any one of embodiments 72 to 76, wherein the genome repair is made by homology directed repair (HDR).
79. The method of any one of embodiments 72 to 76, wherein the genome repair is made by non-homologous end joining (NHEJ) repair.
80. The method of any one of embodiments 72 to 76, wherein the recruiting moiety is streptavidin and the binding moiety is biotin or wherein the recruiting moiety is biotin and the binding moiety is streptavidin.
81. The method of any one of embodiments 72 to 76, wherein the recruiting moiety is Epstein-Barr virus (EBV)-encoded nuclear antigen-1 (EBNA1) and the binding moiety is an origin of plasmid replication (oriP) or wherein the recruiting moiety is oriP and the binding moiety is EBNA1.
82. The method of any one of embodiments 72 to 76, wherein the recruiting moiety is SV40 T-antigen and the binding moiety is SV40 origin of replication or wherein the recruiting moiety is SV40 origin of replication and the binding moiety is SV40 T-antigen.
83. The method of any one of embodiments 72 to 76, wherein the recruiting moiety is BK T-antigen and the binding moiety is BK Virus (BKV) origin of replication or wherein the recruiting moiety is BKV origin of replication and the binding moiety is BK T-antigen.
84. The method of any one of embodiments 72 to 76, wherein the recruiting moiety is latency-associated nuclear antigen (LANA1) of Karposi's Sarcoma Herpesvirus (KSHV) and the binding moiety is LANA binding site (LBS) of KSHV or wherein the recruiting moiety is LANA binding site of KSHV and the binding moiety is LANA1 of KSHV.
85. The method of any one of embodiments 72 to 76, wherein the recruiting moiety is E2 protein of human papilloma virus (HPV) and the binding moiety is minichromosome maintenance element (MME) region of HPV or wherein the recruiting moiety is MME region of HPV and the binding moiety is E2 protein of HPV.
86. The method of any one of embodiments 72 to 76, wherein the recruiting moiety is an HUH-tag and the binding moiety is an HUH recognition sequence or wherein the recruiting moiety is an HUH recognition sequence and the binding moiety is an HUH-tag.
87. The method of any one of embodiments 72 to 76, wherein the recruiting moiety is a Udg variant and the binding moiety is uracilated DNA or wherein the recruiting moiety is uracilated DNA and the binding moiety is a Udg variant.
88. The method of any one of embodiments 72 to 76, wherein the recruiting moiety is a retron and the binding moiety is retron-synthesized RNA.
89. The method of any one of embodiments 72 to 76, wherein the recruiting moiety of the binding pair is positioned between the two orthogonal nucleases.
90. The method of any one of embodiments 72 to 76, wherein the recruiting moiety of the binding pair is positioned N-terminal to the two orthogonal nucleases.
91. The method of any one of embodiments 72 to 76, wherein the recruiting moiety of the binding pair is positioned C-terminal to the two orthogonal nucleases.
92 The method of any one of embodiments 72 to 76, wherein each of the two orthogonal nucleases is a Type II nuclease.
93. The method of any one of embodiments 72 to 76, wherein each of the two orthogonal nucleases is a Type V nuclease.
94. The method of any one of embodiments 72 to 76, wherein the two orthogonal nucleases comprises one Type II nuclease and one Type V nuclease.
95. A method for increasing genome repair during CRISPR editing of a genome in a live cell, comprising:
   a. providing a live cell suitable for editing, wherein the live cell comprises a ribonucleoprotein (RNP) complex formed with a nuclease, a guide RNA, and a repair template, wherein the guide RNA comprises a G-quadruplex (GQ)-forming sequence, wherein the repair template comprises a corresponding GQ-forming sequence;
   b. providing conditions for editing the cell, wherein the guide RNA and the repair template are non-covalently linked in a GQ thereby bringing the repair template and the nuclease into proximity with one another.
96. The method of embodiment 95, wherein the repair template comprises a single-stranded oligonucleotide donor DNA (ssODN).
97 The method of embodiment 95, wherein the GQ-forming sequence is at the 3′ end of the guide RNA.
98. The method of embodiment 95, wherein the corresponding GQ-forming sequence is at either the 3′ end or the 5′ end of the repair template.
99. A method for increasing genome repair during CRISPR editing of a genome in a live cell, comprising:
   a. providing a live cell suitable for editing, wherein the live cell comprises a ribonucleoprotein (RNP) complex formed with a nuclease, a guide RNA, and a repair template, wherein the guide RNA comprises a heteroduplex barcode sequence, wherein the repair template comprises the reverse complement of the heteroduplex barcode sequence;
   b. providing conditions for editing the cell, wherein the guide RNA and the repair template are non-covalently linked in a RNA/DNA hybrid thereby bringing the repair template and the nuclease into proximity with one another.
100. The method of embodiment 99, wherein the repair template comprises a single-stranded oligonucleotide donor DNA (ssODN).
101. The method of embodiment 99, wherein the heteroduplex barcode sequence is about 20 nucleotides in length.
102. The method of embodiment 99, wherein the heteroduplex barcode sequence is at the 3′ end of the guide RNA.
103. The method of embodiment 99, where the heteroduplex barcode sequence is at either the 3′ end or the 5′ end of the repair template.
104. The method of any one of embodiments 95-103, wherein the RNP complex further comprises a binding moiety of a binding pair and a recruiting moiety of the binding pair.

1. A system comprising: (i) a fusion polypeptide comprising first and second orthogonal nucleases and further comprising or coupled to a recruiting moiety, (ii) a repair template comprising or coupled with a binding moiety, and optionally (iii) one or more guide RNAs; wherein the recruiting moiety recognizes the binding moiety and forms a binding pair.

2. The system of claim 1, wherein the system performs genome repair via homology directed repair (HDR).

3. The system of claim 1, wherein one or both of the first and second orthogonal nucleases is an RNA-guided nuclease.

4. The system of claim 1, wherein one or both of the first and second orthogonal nucleases is a CRISPR nuclease.

5. The system of claim 1, wherein the recruiting moiety is streptavidin and the binding moiety is biotin or wherein the recruiting moiety is biotin and the binding moiety is streptavidin.

6. The system of claim 1, wherein the recruiting moiety is Epstein-Barr virus (EBV)-encoded nuclear antigen-1 (EBNA1) and the binding moiety is an origin of plasmid replication (oriP) or wherein the recruiting moiety is oriP and the binding moiety is EBNA1.

7. The system of claim 1, wherein the recruiting moiety is SV40 T-antigen and the binding moiety is SV40 origin of replication or wherein the recruiting moiety is SV40 origin of replication and the binding moiety is SV40 T-antigen.

8. The system of claim 1, wherein the recruiting moiety is BK T-antigen and the binding moiety is BK Virus (BKV) origin of replication or wherein the recruiting moiety is BKV origin of replication and the binding moiety is BK T-antigen.

9. The system of claim 1, wherein the recruiting moiety is latency-associated nuclear antigen (LANA1) of Karposi's Sarcoma Herpesvirus (KSHV) and the binding moiety is LANA binding site (LBS) of KSHV or wherein the recruiting moiety is LANA binding site of KSHV and the binding moiety is LANA1 of KSHV.

10. The system of claim 1, wherein the recruiting moiety is E2 protein of human papilloma virus (HPV) and the binding moiety is minichromosome maintenance element (MME) region of HPV or wherein the recruiting moiety is MME region of HPV and the binding moiety is E2 protein of HPV.

11. The system of claim 1, wherein the recruiting moiety is an HUH-tag and the binding moiety is an HUH recognition sequence or wherein the recruiting moiety is an HUH recognition sequence and the binding moiety is an HUH-tag.

12. The system of claim 1, wherein the recruiting moiety is a Udg variant and the binding moiety is uracilated DNA or wherein the recruiting moiety is uracilated DNA and the binding moiety is a Udg variant.

13. The system of claim 1, wherein the recruiting moiety is a retron and the binding moiety is retron-synthesized RNA.

14. The system of claim 1, wherein the one or more guide RNAs comprise a G-quadruplex (GQ)-forming sequence, wherein the repair template further comprises a corresponding GQ-forming sequence, and wherein the one or more guide RNAs is non-covalently linked to the repair template by formation of a GQ.

15. The system of claim 1, wherein the one or more guide RNAs comprise a heteroduplex barcode sequence, wherein the repair template further comprises the reverse complement of the heteroduplex barcode sequence, and wherein the one or more guide RNA is non-covalently linked to the repair template by formation of a RNA/DNA hybrid.

16. A system comprising: (i) an RNA-guided nuclease, (ii) a guide RNA comprising or coupled to a recruiting moiety, and (iii) a repair template molecule comprising or coupled with a binding moiety; wherein the recruiting moiety recognizes the binding moiety and forms a binding pair.

17. The system of claim 16, wherein the recruiting moiety comprises a G-quadruplex (GQ)-forming sequence.

18. The system of claim 17, wherein the binding moiety comprises a corresponding G-quadruplex (GQ)-forming sequence.

19. The system of claim 16, wherein the recruiting moiety comprises a heteroduplex barcode sequence.

20. The system of claim 19, wherein the binding moiety comprises a reverse complement of the heteroduplex barcode sequence.


SEQUENCE LISTING (5 sequences)

SEQ ID NO: 1 - skipped

SEQ ID NO: 2 - RNA, 12 nt, synthetic construct
  source 1..12: mol_type = other RNA
tagggttagggt

SEQ ID NO: 3 - DNA, 12 bp, synthetic construct
  source 1..12: mol_type = other DNA
tagggttagggt

SEQ ID NO: 4-5 - skipped (2 entries)